(19) United States
(12) Patent Application Publication
(10) Pub. No.: US 2017/0107503 A1
(43) Pub. Date: Apr. 20, 2017

(54) NOVEL METHODS, POLYPEPTIDES AND USES THEREOF

(71) Applicant: ENZYMATICA AB, Lund (SE)

(72) Inventors: Mats Peter CLARSUND, Lund (SE); Bo Roger SVENSSON, Södra Sandby (SE); Björn Ulrik WALSE, Lund (SE); Poul Baad RASMUSSEN, Dyssegård (DK); Peterø Bjødstrup THORSTED, Tikøb (DK)

(21) Appl. No.: 15/300,567

(22) PCT Filed: Mar. 31, 2015
(86) PCT No.: PCT/GB2015/051006

§ 371 Date: Sep. 29, 2016

(30) Foreign Application Priority Data
Mar. 31, 2014 (GB) 1405784.8

(51) Int. Cl. C12N 9/76; A61Q 19/08; A61K 8/66; A61Q 7/00; A61K 38/48; A61K 9/00

(52) U.S. Cl. CPC C12N 9/6427; C12Y 304/21004; A61K 38/4826; A61K 9/006; A61K 8/66; A61Q 7/00; A61Q 19/08; A61K 2800/28

(57) ABSTRACT

The present invention provides methods for the production of recombinant polypeptides having serine protease activity, polypeptides obtainable by such methods and use of said polypeptides in medicine, cosmetics and industry. In particular, the invention provides recombinantly expressed mutants of trypsin I from Atlantic cod, which mutants exhibit improved stability and/or catalytic properties relative to the wildtype trypsin purified from cod.

### FIELD OF INVENTION

The present invention relates to methods for the production of recombinant polypeptides having serine protease activity, polypeptides obtainable by such methods and use of said polypeptides in medicine, cosmetics and industry.

### BACKGROUND

Enzymes that cleave peptide bonds in proteins are also known as proteases, proteinases, peptidases, or proteolytic enzymes [1], and function to accelerate the rate of specific biologic reactions by lowering the activation energy of the reaction [2]. Proteases are most often assumed only to be involved in processes relating to digestion, but the fact that over 2% of the human genome encodes protease genes suggests that they play more complex functions than digestion alone [3]. Indeed, proteases have been shown to be involved in the regulation of a number of cellular components from growth factors to receptors, as well as processes including immunity, complement cascades, and blood coagulation [3]. In addition to involvement in homeostatic processes, increased or dysregulated activity of proteases has been implicated in cancer via its link with tumour growth and invasion [4].

Briefly, proteases are initially produced as inactive precursors, or zymogens, and are distributed in specific organs or locations, where they have little catalytic ability until they are activated by proteolytic cleavage [5]. Further posttranslational mechanisms to control the activity of proteases include phosphorylation, cofactor binding, and segregation of enzyme and/or substrate in vesicles or granules. In addition, the effective concentration of active enzyme can also be strictly regulated by protease inhibitors, which can reduce functional efficacy by forming a complex with the protease and effectively “balance” proteolytic activity.

Proteases have been used in medicine for several decades and are an established and well tolerated class of therapeutic agent [3]. Early documented use of proteases in the published literature appeared over 100 years ago [7-9]. In general, proteases have been used in four areas: the management of gastrointestinal disorders with orally administered agents, as anti-inflammatory agents, as thrombolytic agents for thromboembolic disorders, and as locally administered agents for wound debridement [10]. Since the first approval of a protease drug in 1978 (urokinase, a serine protease indicated for thrombolysis and catheter clearing), a further 11 drugs have been approved for therapeutic use by the US Food and Drug Administration (FDA) [3]. The majority of these are indicated for the treatment of blood disorders and include thrombolytics: alteplase, reteplase, and tenecteplase; and procoagulants: factor IX, factor VIIa, thrombin, and topical thrombin in bandages. The other approved protease therapeutics are indicated for digestion (pancrelipase), muscle spasms, and as cosmeceuticals (cosmetic products with biologically active ingredients intended to have medicinal or drug-like benefits; botulinum toxin A and botulinum toxin B) [3].

The proteases so far approved by the US FDA are sourced from a range of mammals or bacteria that exist or have adapted to moderate temperatures, i.e. mesophilic organisms. In the pursuit of more effective and more flexible proteases, the therapeutic potential of molecules derived from organisms from cold environments has been examined. Those organisms from the three domains of life (bacteria, archaea, eucarya) that thrive in cold environments (i.e. psychrophiles) have developed enzymes that generally have high specific activity, low substrate affinity, and high catalytic rates at low and moderate temperatures [18-20]. In general, when compared with mesophilic variants, the property of greater flexibility in psychrophilic enzymes allows the protease to interact with and transform the substrate at lower energy costs. The comparative ease of interaction is possible because the catalytic site of the psychrophilic protease can accommodate the substrate more easily [20]. However, this increased flexibility is often accompanied by a trade-off in stability [21]. Therefore, in contrast to mammalian analogs, psychrophilic proteases are more sensitive to inactivation by heat, low pH, and autolysis [18, 19, 21-25].

While psychrophilic proteases have been obtained from biological sources, such as Atlantic cod (Gadus morhua) or Antarctic krill (Euphausia superba), the large-scale production of suitable quantities of homogenous coldadapted proteases could be obtained using recombinant technologies. A wide variety of fish enzymes and proteases has already been identified, cloned, and expressed in microorganisms [36]. In the production of other proteases for therapeutic purposes, non-human sources or production hosts are preferred so that the potential for contamination can be avoided. Recombinant technologies are thus widely employed to produce approved mammalian (recombinant) therapeutic proteins, such as blood clotting factors (from recombinant Chinese hamster ovary or baby hamster kidney cells), thrombolytics (from Escherichia coli), or botulinum toxin (Clostridium botulinum)[3]. Therefore, it would appear desirable to explore the possibility of producing cold-adapted proteases through recombinant technology. There have been several, more or less successful, attempts to do this in the laboratory. However, large-scale production of recombinant cold-adapted enzymes is associated with several complicating factors, such as the short half-life and autolytic activity of cold-adapted enzymes, which makes production difficult under more standardized industrial conditions and temperatures.

The present inventions seeks to overcome these problems by providing a method for the production of recombinant serine protease polypeptides, such as cold-adapted trypsins, which is suitable for large-scale production.

The invention further seeks to provide mutant serine protease polypeptides with improved properties, such as stability and catalytic activity, compared to serine proteases purified from natural sources.

### SUMMARY OF INVENTION

The first aspect of the invention provides a method for the production of a recombinant polypeptide having serine protease activity comprising
(a) transforming a microbial host cell, or population thereof, with a nucleic acid molecule encoding a zymogen polypeptide comprising an activation peptide fused to the N-terminus of a polypeptide having serine protease activity
wherein the zymogen polypeptide lacks a signal sequence;
(b) expressing said zymogen polypeptide in the host cell(s) as inclusion bodies;
(c) purifying the zymogen polypeptide from the host cell(s); and
(d) activating the zymogen polypeptide by exposure to a protease, such as a trypsin
wherein step (c) comprises solubilising the zymogen polypeptide from the inclusion bodies and refolding the polypeptide into a bioactive form.

Thus, the invention provides an in vitro method for the production of recombinant polypeptide having serine protease activity

By polypeptide having serine protease activity we include both naturally occurring and non-naturally occurring catalytic polypeptides capable of cleaving peptide bonds in proteins, in which serine serves as the nucleophilic amino acid at the active site of the polypeptide (as defined in accordance with EC Number 3.4.21). The serine protease activity may be chymotrypsin-like (i.e. trypsins, chymotrypsins and elastases) or subtilisin-like.

In one embodiment, the polypeptide having serine protease activity exhibits trypsin activity. For example, the polypeptide having serine protease activity may be a naturally-occurring trypsin, of either eukaryotic or prokaryotic origin, or a mutated version of such a trypsin. Specifically included are cold-adapted trypsins, such as a trypsin from Atlantic cod (Gadus morhua), Atlantic and Pacific salmon (e.g. Salmo salar and species of Oncorhynchus) and Alaskan Pollock (Theragra chalcogramma), and mutated forms thereof (as described in detail below).

Three major isozymes of trypsin have been characterised from Atlantic cod, designated Trypsin I, II and III (see Asgeirsson et al., 1989, Eur. J. Biochem. 180:85-94, the disclosures of which are incorporated herein by reference). For example, see GenBank Accession No. AC090397.

In addition, Atlantic cod expresses two major isozymes of chymotrypsin, designated Chymotrypsin A and B (see Asgeirsson & Bjarnason, 1991, Comp. Biochem. Physiol. B 998:327-335, the disclosures of which are incorporated herein by reference). For example, see GenBank Accession No. CAA55242.1.

In one embodiment, the polypeptide having serine protease activity comprises or consists of an amino acid sequence which shares at least 70% sequence identity with amino acid sequence of trypsin I from Atlantic cod (Gadus morhua), i.e. SEQ ID NO: 1:

```
[SEQ ID NO: 1]
  16
I
IVGGYECTKHSQAHQVSLNSGYHFCGGSLVSKDWVVSAAHCYKSVLRVRL
GEHHIRVNEG
  79
I
TEQYISSSSVIRHPNYSSYNINNDIMLIKLTKPATLNQYVHAVALPTECA
ADATMCTVSG
141
I
WGNTMSSVADGDKLQCLSLPILSHADCANSYPGMITQSMFCAGYLEGGKD
SCQGDSGGPV
200
I
VCNGVLQGVVSWGYGCAERDHPGVYAKVCVLSGWVRDTMANY
```

(wherein the amino acid sequence and numbering is according to Protein Data Bank [PDB] entry ‘2EEK!’)

Like many proteases, trypsin I from Atlantic cod is produced as an inactive precursor, or zymogen, comprising a propeptide (or “activation”) sequence that is cleaved off to generate the mature, active trypsin. The initial expression product for trypsin also comprises a signal sequence, which is removed following expression.

The zymogen sequence for trypsin I from Atlantic cod, including the signal sequence is shown below as SEQ ID NO:2 (and corresponds to Uniprot database accession no. P16049-1):

```
[SEQ ID NO: 2]
                10 20 30 40
MKSLIFVLLL GAV [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

wherein:

Signal peptide=amino acids 1 to 13 (underlined)

Propeptide=amino acids 14 to 19 (bold italics) Mature trypsin=amino acids 20 to 241

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g., α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the polypeptides shown, each encoded amino acid residue, where appropriate, is represented by a single letter designation, corresponding to the trivial name of the conventional amino acid.

In accordance with convention, the amino acid sequences disclosed herein are provided in the N-terminus to C-terminus direction.

Typically, the polypeptides used in the compositions of the invention comprise or consist of L-amino acids.

The polypeptide having serine protease activity may comprise or consist of an amino acid sequence which shares at least 80%, 85%, 90%, 95%, 95%, 97%, 98% or 99% sequence identity with SEQ ID NO:1.

Thus, in one embodiment, the polypeptide having serine protease activity may comprise or consist of the amino acid sequence of SEQ ID NO:1.

However, the polypeptide may alternatively comprise or consist of the amino acid sequence which is a mutant or variant of SEQ ID NO:1. By “variant” we mean that the polypeptide does not share 100% amino acid sequence identity with SEQ ID NO: 1, i.e. one or more amino acids of SEQ ID NO: 1 must be mutated. For example, the polypeptide may comprise or consist of an amino acid sequence with at least 50% identity to the amino acid sequence of SEQ ID NO: 1, more preferably at least 60%, 70% or 80% or 85% or 90% identity to said sequence, and most preferably at least 95%, 96%, 97%, 98% or 99% identity to said amino acid sequence. Thus, an amino acid at a specified position may be deleted, substituted or may be the site of an insertion/addition of one or more amino acids. It will be appreciated by persons skilled in the art that the substitutions may be conservative or non-conservative.

Percent identity can be determined by, for example, the LALIGN program (Huang and Miller, Adv. Appl. Math. (1991) 12:337-357, the disclosures of which are incorporated herein by reference) at the Expasy facility site (http://www.ch.embnet.org/software/LALIGN_form.html) using as parameters the global alignment option, scoring matrix BLOSUM62, opening gap penalty −14, extending gap penalty −4. Alternatively, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (as described in Thompson et al., 1994, Nucl. Acid Res. 22:4673-4680, which is incorporated herein by reference). The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

Thus, the polypeptide having serine protease activity may be a variant of SEQ ID NO:1

In one embodiment, the polypeptide having serine protease activity is a variant of SEQ ID NO:1 or 2 comprising one or more mutated amino acids selected from the group consisting of amino acid positions (wherein the same mutation sites may be defined by reference to two alternative numbering systems):
Defined by reference to Protein Data Bank [PDB] entry 2EEK! (SEQ ID NO:1):
E21, H25, H29, V47, K49, D50, L63, H71, H72, R74, N76, T79, Y82, S85, S87, N98, 199, V121, M135, V138, M145, V148, D150, K154, L160, M175, S179, A183, L185, V212, Y217, P225, A229, V233, L234, V238, M242, N244, and/or Y245.
Defined by reference to Uniprot entry. P16049-1 9 (SEQ ID NO:2):
E25, H29, H33, V49, K51, D52, L65, H72, H73, R75, N77, T80, Y83, S86, S88, N99, 1100, V122, M134, V137, M144, V147, D149, K152, L158, M173, S177, A181, L184, V208, Y213, P221, A225, V229, L230, V234, M238, N240, and/or Y241.

Thus, the polypeptide having serine protease activity may be a variant of SEQ ID NO:1 comprising one or more amino acids mutations selected from the group consisting of:
Defined by reference to Protein Data Bank [PDB] entry 2EEK! (SEQ ID NO:1):
E21T, H25Y, H29(Y/N), V47I, K49E, D50Q, L63I, H71D, H72N, R74(K/E), N76(T/L), T79(S/N), Y82F, S85A, S87(K/R), S89R, N98T, I99L, V121I, M135Q, V138I, M145(T/L/V/E/K), V148G, D150S, K154(T/V), L160(I/A), M175(K/Q), S179N, A183V, L185G, V212I, Y217(D/H/S), P225Y, A229V, V233N, L234Y, V238I, M242I, N244S, and/or Y245N.
Defined by reference to Uniprot entry. P16049-1 and (SEQ ID NO:2):
E25T, H29Y, H33(Y/N), V49I, K51E, D52Q, L65I, H72D, H73N, R75(K/E), N77(T/L), T80(S/N), Y83F, S86A, S88(K/R), N99T, 1100L, V122I, M134Q, V137I, M144(T/UV/E/K), V147G, D149S, K152(TN), L158(I/A), M173(K/Q), S177N, A181V, L184G, V208I, Y213(D/H/S), P221Y, A225V, V229N, L230Y, V234I, M238I, N240S, and/or Y241N.

For example, the polypeptide having serine protease activity may comprise or consist of the amino acid sequence of SEQ ID NO:1 with one of the following defined mutations or combinations thereof (Table 1):

TABLE 1

| | Mutation(s) in SEQ ID | Mutation(s) in SEQ ID |
|---|---|---|
| ID number | NO: 1 (PDB 2EEK!) | NO: 2 (UniProt P16049-1) |
| EZA-001 | Wildtype | Wildtype |
| EZA-002 | N244S, Y245N, S87K | N240S, Y241N, S88K |
| EZA-003 | K154T | K152T |
| EZA-004 | K154L | K152L |
| EZA-005 | K154V | K152V |
| EZA-006 | K154E | K152E |
| EZA-007 | N98T | N99T |
| EZA-008 | I99L | I100L |
| EZA-009 | L185G, P225Y | L184G, P221Y |
| EZA-010 | V212I | V208I |
| EZA-011 | Y217D, M175K | Y213D, M173K |
| EZA-012 | Y217H | Y213H |
| EZA-013 | Y217S | Y213S |
| EZA-014 | A229V | A225V |
| EZA-015 | H25Y | H29Y |
| EZA-016 | H25N | H29N |
| EZA-017 | H29Y | H33Y |
| EZA-018 | H71D | H72D |
| EZA-019 | H72N | H73N |
| EZA-020 | R74K | R75K |
| EZA-021 | R74E | R75E |
| EZA-022 | N76T | N77T |
| EZA-023 | N76L, Y82F | N77L, Y83F |
| EZA-024 | T79S | T80S |
| EZA-025 | T79N | T80N |
| EZA-026 | K49E, D50Q | K51E, D52Q |
| EZA-027 | S87R | S88K |
| EZA-028 | E21T, H71D, D150S, | E25T, H72D, D149S, K152V |
| | K154V | |
| EZA-029 | S179N, V233N | S177N, V229N |
| EZA-030 | M135Q | M134Q |
| EZA-031 | M145K, V148G | M144K, V147G |
| EZA-032 | M175Q | M173Q |
| EZA-033 | L63I, S85A | L65I, S86A |
| EZA-034 | L160I | L158I |
| EZA-035 | V138I, L160A, A183V | V137I, L158A, A181V |
| EZA-036 | V121I | V122I |
| EZA-037 | V47I, V238I, M242I | V49I, V234I, M238I |
| EZA-038 | V238I | V234I |
| EZA-039 | L234Y | L230Y |

Likewise, the polypeptide having serine protease activity may comprise or consist of the amino acid of SEQ ID NO:1 with one of the following defined mutations or combinations thereof (Table 2):

TABLE 2

| Mutation(s) in SEQ ID | Mutation(s) in SEQ ID |
|---|---|
| NO: 1 (PDB 2EEK!) | NO: 2 (UniProt P16049-1) |
| H25N, N76T | H29N, N77T |
| H25N, H29Y | H29N, H33Y |
| H25N, M135Q | H29N, M134Q |
| H29Y, T79N, M135Q | H33Y, T80N, M134Q |
| I99L, V121I, L160I, Y217H | I100L, V122I, L158I, |
| | Y213H |
| V121I, L160I | V122I, L158I |
| H72N, R74E, S87K | H73N, R75E, S88K |
| H25N, M135Q, Y217H | H29N, M134Q, Y213H |
| T79N, V121I, V212I | T80N, V122I, V208I |
| H29Y, N76T, I99L, M135Q | H33Y, N77T, I100L, |
| | M134Q |
| K49E, D50Q, N76L, Y82F, | K51E, D52Q, N77L, Y83F, |
| S179N, V233N | S177N, V229N |
| M145K, V148G, N76L, | M144K, V147G, N77L, |
| Y82F, S179N, V233N | Y83F, S177N, V229N |
| H25N, N76T, S87K, | H29N, N77T, S88K, |
| K154T | K152T |
| H25Q | H29Q |
| H25D | H29D |
| H25S | H29S |
| K24E, H25N | K28E, H29N |
| Y97N | Y98N |
| N100D | N101D |
| A120S, A122S | A121S, A123S |
| M135E | M134E |
| V204Q, A122S | V203Q, A123S |
| T79D | T80D |
| R74D | R75D |
| K49E | K51E |
| K49S, D50Q | K51S, D52Q |
| D50Q | D52Q |
| Q178D | Q176D |
| S87R | S88R |

In Tables 1 and 2 above, where the polypeptide having serine protease activity is defined by reference to mutation(s) in SEQ ID NO:2 (i.e. UniProt P16049-1), it will be appreciated that the mature protease will commence with 120 as its N-terminal amino acid. However, it will typically be expressed initially as a zymogen polypeptide having an activation sequence at its N-terminus (see below).

In one preferred embodiment, the polypeptide having serine protease activity is a variant of the amino acid sequence of SEQ ID NO:1 which does not comprise histidine at position 25.

For example, the polypeptide having serine protease activity may comprise or consist of the amino acid sequence of SEQ ID NO:3 (“EZA-016” in Table 1, comprising an H25N mutation; see box in sequence below):

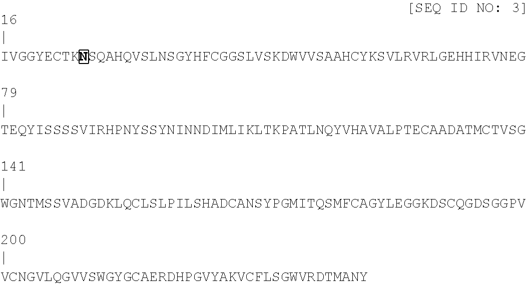

In an alternative preferred embodiment, the polypeptide having serine protease activity is a variant of the amino acid sequence of SEQ ID NO:1 which does not comprise lysine at position 160.

For example, the polypeptide having serine protease activity may comprise or consist of the amino acid sequence of SEQ ID NO:4 (“EZA-034” in Table 1, comprising an L160I mutation; see box in sequence below):

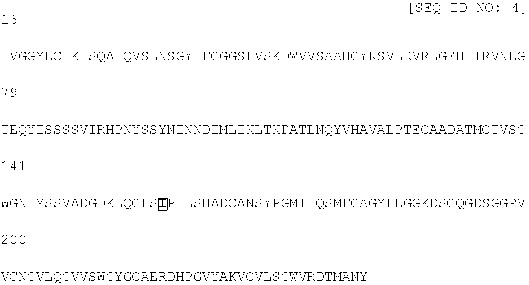

It will be appreciated by persons skilled in the art that the above identified mutations (defined by reference to the amino acid sequence of trypsin I of Atlantic cod, SEQ ID NO:1) could also be made in trypsins from other species. For example, the specific mutations highlighted in SEQ ID NOS: 3 and 4 (H25N and L160I with reference to SEQ ID NO:1 and PDB 2EEK!), respectively) could be made in the trypsin from Alaskan Pollock (for example see GenBank: BAH70476.3, wherein the amino acid sequence of the active trypsin commences at position 120, such that H25 corresponds to H29 in BAH70476.3, etc).

In an alternative embodiment, the polypeptide having serine protease activity comprises or consists of the amino acid sequence of a naturally-occurring serine protease. Thus, the polypeptide having serine protease activity may consist of the amino acid sequence of a naturally-occurring trypsin, of either eukaryotic or prokaryotic origin. Specifically included are cold-adapted trypsins, such as a trypsin from Atlantic cod (Gadus morhua), Atlantic and Pacific salmon (e.g. Salmo salar and species of Oncorhynchus) and Alaskan Pollock (Theragra chalcogramma).

For example, the polypeptide having serine protease activity may comprise or consist of the amino acid of SEQ ID NO:1 (i.e. as shown in Protein Data Bank entry 2EEK!).

It will be appreciated by persons skilled in the art that the polypeptide having serine protease activity may also comprise or consist of a fragment of any of the above defined amino acid sequences, wherein the fragment exhibits an antimicrobial (for example, antibacterial) activity.

By “fragment” we include at least 5 contiguous amino acids of any of the above amino acid sequences, such as but not limited to SEQ ID NO: 1, 2, 3 or 4. For example, the fragment may comprise at least 10, 11, 12, 13, 14, 15, 16, 17, 18, 19, 20, 25, 30, 35, 40, 45, 50, 60, 70, 80, 90, 100, 120, 140, 160, 180, 200 or more contiguous amino acids of any of the above amino acid sequences.

Methods of identifying fragments of the above-defined serine protease polypeptides which retain an antimicrobial (for example, antibacterial) activity are well known in the art. For example, a range of different fragments could be generated known recombinant methodologies, using the expression methods of the invention, and then exposed in vitro to representative microorganisms (such as bacterial strains, viruses and/or fungal strains) to determine which of the fragments inhibits (in part or in whole) the growth and/or proliferation of said microorganisms.

For example, in trypsin I from Atlantic cod (SEQ ID NO:1), the regions highlighted below are believed to retain the antibacterial properties of the parent protein:

```
[SEQ ID NO: 1]
  16
I
IVGGYECTKHSQAHQVSLNSGYHFCGGSLVSKDWVVSAAHC [symbol] [symbol]
EG
  79
I
TEQYISSSSVIRHPNYSSYNINNDIMLIKLTKPATLNQYVHAVALPTECA
ADATMCIVSG
141
I
WGNTMSSVADGDKLQCLSLPILSHADCANSYPGMITQSMFCAGYLEGGKD
SCQGDSGGPV
200
I
VCNGVLQGVVSWGYGCAERDHPGVY [symbol] [symbol]
```

Corresponding regions exhibiting antibacterial activity may be identified in any of the other amino acid sequences described herein.

Development of the methods of the present invention was the culmination of extensive efforts over a prolonged period (18 months) to overcome difficulties in producing trypsin-like enzymes by recombinant means. Only after several failed attempts did the inventors devise the present methodology, in which polypeptides having serine protease activity are produced by expression as inclusion bodies without a signal peptide and subsequent refolding of the active polypeptide.

A characterising feature of the methods of the invention is the step of expressing in a microbial host cell a zymogen polypeptide comprising an activation peptide fused to the N-terminus of a polypeptide having serine protease activity (wherein the zymogen polypeptide lacks a signal sequence).

By “zymogen polypeptide” we mean an inactive precursor form (‘pro-enzyme’) of the polypeptide having serine protease activity, which may subsequently be proteolytically cleaved to release the active serine protease polypeptide. For example, where the polypeptide having serine protease activity is a trypsin, the zymogen polypeptide is a trypsinogen.

By “activation peptide” we mean a short peptide (typically four or five amino acids in length) which is released upon activation of the zymogen by exposure to a protease, such as a trypsin (see Chen, Jian-Min, et al. “Evolution of trypsinogen activation peptides.” Molecular biology and evolution 20.11 (2003): 1767-1777, the disclosures of which are incorporated herein by reference). It will be appreciated that the activation peptides may be naturally-occurring activation peptides or mutated versions of the same.

In one embodiment, wherein the activation peptide comprises or consist of the amino acid sequence selected from the following group:

[SEQ ID NO: 5]
(a) MEEDK;
[SEQ ID NO: 6]
(b) MTEEDK;
[SEQ ID NO: 7]
(c) MFAEEDK;
[SEQ ID NO: 8]
(d) MVFAEEDK;
[SEQ ID NO: 9]
(e) MAFAEEDK;
and
[SEQ ID NO: 10]
(f) MGAVFAEEDK.

The nucleic acid molecule encoding the zymogen polypeptide is inserted in an expression vector appropriate for expression of recombinant proteins in the selected host cell type (see below).

Expression vectors suitable for use in microbial host cells are widely available commercially (from companies such as Novagen, Invitrogen, Qiagen, Stratagene and GenScript).

In one embodiment, the nucleic acid molecule encoding a trypsinogen polypeptide is in an expression vector suitable for use in Escherichia coli, such as expression vector E3 (available from GenScript USA Inc, Piscataway, USA).

It will be appreciated by persons skilled in the art that the methods of the invention may be performed using any suitable microbial cell as a host cell, for example bacterial cells, fungal cells and yeast cells.

In one preferred embodiment, the host cell in step (a) is a bacterial host cell (such as Escherichia coli and Pseudoalteromonas haloplanktis). For example, the host cell in step (a) may be an Escherichia coli host cell (such as BL21(E3), BL21(DE3), BL21 Star (DE3), ArcticExpress (DE3) and HMS174 cells).=

In an alternative embodiment, the host cell in step (a) is a yeast host cell (such as Pichia pastoris).

Once transformed with the expression vector, the host cells are cultured under conditions suitable to induce expression of the zymogen polypeptide. Culture conditions and media for different types of microbial cells are well known in the art (for example, see Green & Sambrook, 2012, Molecular Cloning, A Laboratory Manual, Fourth Edition, Cold Spring Harbor, N.Y., the relevant disclosures in which document are hereby incorporated by reference).

Thus, in step (b) the host cells may be cultured at a temperature of at least 18° C., for example at 18° C., 22° C., 28° C. or 37° C.

The duration of expression step (b) may be at least 6 hours, for example 8 hours, 16 hours, 24 hours or more.

Where host cells such as BL21(DE3), BL.21 Star (DE3), and ArcticExpress (DE3) are utilised, expression may be induced by an agent such as IPTG (e.g. at 1 mM).

In step (c) of the methods of the invention, the expressed zymogen polypeptide is purified by solubilising and refolding the inclusion body polypeptide. Again, suitable methods for such purification are well known in the art (for example, see Singh & Panda, 2005, J. Biosci. Bioeng. 99(4):303-10 and Burgess, 2009, Methods Enzymol. 463:259-82, the relevant disclosures in which documents are hereby incorporated by reference).

In one embodiment, refolding the polypeptide comprises contacting the polypeptide with a PBS/glycerol buffer (for example, 1×PBS, 10% glycerol, pH 7.4).

The methods of the present invention are advantageous in that they do not require, during the solubilising and refolding step, the inclusion of an inhibitor of autoproteolysis (such as benzamidine). This, in one embodiment, no inhibitor of autoproteolysis is present in step (c).

Following solubilisation and refolding, the purified zymogen polypeptide is proteolytically activated by exposure to a trypsin (which cleaves the activation peptide to reveal the active serine protease polypeptide).

In one embodiment, trypsin from Atlantic cod is used to activate the zymogen polypeptide in step (d).

The methods of the invention are able to produce recombinant serine protease polypeptides having a high specific activity. For example, the specific activity of the activated polypeptide produced in step (d) may be at least 20 U/mg, for example at least 30 U/mg, 40 U/mg, 50 U/mg, or at least 60 U/mg.

The methods of the invention are able to produce good yields of recombinant serine protease polypeptides. For example, the quantity of the activated polypeptide produced in step (d) may be at least 0.1 mg, for example at least 0.5 mg, 1 mg, 2 mg, 3 mg, 5 mg, or 10 mg.

A second aspect of the invention provides an isolated polypeptide having serine protease activity obtainable by a method of the invention (as detailed above).

By “isolated” we mean that the polypeptide is not located or otherwise provided within a cell. Thus, the polypeptide may be provided as a cell-free preparation.

Preferred embodiments of the polypeptides of the invention are described above in relation to the methods of the invention. Thus, the polypeptide having serine protease activity may exhibit trypsin activity.

For example, the polypeptide may comprise or consist of an amino acid sequence which shares at least 70% sequence identity with amino acid sequence of SEQ ID NO:1, for example at least 80%, 85%, 90%, 95%, 95%, 97%, 98% or 99% sequence identity (such as SEQ ID NOS: 3 and 4).

In one embodiment, the polypeptide having serine protease activity is a variant of SEQ ID NO:1 or 2 comprising one or more mutated amino acids selected from the group consisting of amino acid positions (wherein the same mutation sites are defined by reference to two alternative numbering systems):
Defined by reference to Protein Data Bank [PDB] entry 2EEK!:
E21, H25, H29, V47, K49, D50, L63, H71, H72, R74, N76, T79, Y82, S85, S87, N98, 199, V121, M135, V138, M145, V148, D150, K154, L160, M175, S179, A183, L185, V212, Y217, P225, A229, V233, L234, V238, M242, N244, and/or Y245.
Defined by reference to Uniprot entry. P16049-1 9 and SEQ ID NO:2):
E25, H29, H33, V49, K51, D52, L65, H72, H73, R75, N77, T80, Y83, S86, S88, N99, 1100, V122, M134, V137, M144, V147, D149, K152, L158, M173, S177, A181, L184, V208, Y213, P221, A225, V229, L230, V234, M238, N240, and/or Y241.

Thus, the polypeptide having serine protease activity may be a variant of SEQ ID NO:1 comprising one or more amino acids mutations selected from the group consisting of:
Defined by reference to Protein Data Bank [PDB] entry 2EEK!:
E21T, H25Y, H29(Y/N), V47I, K49E, D50Q, L63I, H71D, H72N, R74(K/E), N76(T/L), T79(S/N), Y82F, S85A, S87(K/R), S89R, N98T, I99L, V121I, M135Q, V138I, M145(T/LN/E/K), V148G, D150S, K154(T/V), L160(I/A), M175(K/Q), S179N, A183V, L185G, V212I, Y217(D/H/S), P225Y, A229V, V233N, L234Y, V238I, M242I, N244S, and/or Y245N.
Defined by reference to Uniprot entry. P16049-1 and SEQ ID NO:2):
E25T, H29Y, H33(Y/N), V49I, K51E, D52Q, L65I, H72D, H73N, R75(K/E), N77(T/L), T80(S/N), Y83F, S86A, S88(K/R), N99T, I100L, V122I, M134Q, V137I, M144(T/LN/E/K), V147G, D149S, K152(TN), L158(I/A), M173(K/Q), S177N, A181V, L184G, V208I, Y213(D/H/S), P221Y, A225V, V229N, L230Y, V234I, M238I, N240S, and/or Y241N.

For example, the polypeptide having serine protease activity may comprise or consist of the amino acid sequence of SEQ ID NO:1 with one of the following defined mutations or combinations thereof (Table 1):

TABLE 1

| | Mutation(s) in SEQ ID | Mutation(s) in SEQ ID |
|---|---|---|
| ID number | NO: 1 (PDB 2EEK!) | NO: 2 (UniProt P16049-1) |
| EZA-001 | Wildtype | Wildtype |
| EZA-002 | N244S, Y245N, S87K | N240S, Y241N, S88K |
| EZA-003 | K154T | K152T |
| EZA-004 | K154L | K152L |
| EZA-005 | K154V | K152V |
| EZA-006 | K154E | K152E |
| EZA-007 | N98T | N99T |
| EZA-008 | I99L | I100L |
| EZA-009 | L185G, P225Y | L184G, P221Y |
| EZA-010 | V212I | V208I |
| EZA-011 | Y217D, M175K | Y213D, M173K |
| EZA-012 | Y217H | Y213H |
| EZA-013 | Y217S | Y213S |
| EZA-014 | A229V | A225V |
| EZA-015 | H25Y | H29Y |
| EZA-016 | H25N | H29N |
| EZA-017 | H29Y | H33Y |
| EZA-018 | H71D | H72D |
| EZA-019 | H72N | H73N |
| EZA-020 | R74K | R75K |
| EZA-021 | R74E | R75E |
| EZA-022 | N76T | N77T |
| EZA-023 | N76L, Y82F | N77L, Y83F |
| EZA-024 | T79S | T80S |
| EZA-025 | T79N | T80N |
| EZA-026 | K49E, D50Q | K51E, D52Q |
| EZA-027 | S87R | S88K |
| EZA-028 | E21T, H71D, D150S, | E25T, H72D, D149S, K152V |
| | K154V | |
| EZA-029 | S179N, V233N | S177N, V229N |
| EZA-030 | M135Q | M134Q |
| EZA-031 | M145K, V148G | M144K, V147G |
| EZA-032 | M175Q | M173Q |
| EZA-033 | L63I, S85A | L65I, S86A |
| EZA-034 | L160I | L158I |
| EZA-035 | V138I, L160A, A183V | V137I, L158A, A181V |
| EZA-036 | V121I | V122I |
| EZA-037 | V47I, V238I, M242I | V49I, V234I, M238I |
| EZA-038 | V238I | V234I |
| EZA-039 | L234Y | L230Y |

Likewise, the polypeptide having serine protease activity may comprise or consist of the amino acid of SEQ ID NO:1 with one of the following defined mutations or combinations thereof (Table 2):

TABLE 2

| Mutation(s) in SEQ ID | Mutation(s) in SEQ ID |
|---|---|
| NO: 1 (PDB 2EEK!) | NO: 2 (UniProt P16049-1) |
| H25N, N76T | H29N, N77T |
| H25N, H29Y | H29N, H33Y |
| H25N, M135Q | H29N, M134Q |
| H29Y, T79N, M135Q | H33Y, T80N, M134Q |
| I99L, V121I, L160I, Y217H | I100L, V122I, L158I, |
| | Y213H |
| V121I, L160I | V122I, L158I |
| H72N, R74E, S87K | H73N, R75E, S88K |
| H25N, M135Q, Y217H | H29N, M134Q, Y213H |
| T79N, V121I, V212I | T80N, V122I, V208I |
| H29Y, N76T, I99L, M135Q | H33Y, N77T, I100L, |
| | M134Q |
| K49E, D50Q, N76L, Y82F, | K51E, D52Q, N77L, Y83F, |
| S179N, V233N | S177N, V229N |
| M145K, V148G, N76L, | M144K, V147G, N77L, |
| Y82F, S179N, V233N | Y83F, S177N, V229N |
| H25N, N76T, S87K, | H29N, N77T, S88K, |
| K154T | K152T |
| H25Q | H29Q |
| H25D | H29D |
| H25S | H29S |
| K24E, H25N | K28E, H29N |
| Y97N | Y98N |
| N100D | N101D |
| A120S, A122S | A121S, A123S |
| M135E | M134E |
| V204Q, A122S | V203Q, A123S |
| T79D | T80D |
| R74D | R75D |
| K49E | K51E |
| K49S, D50Q | K51S, D52Q |
| D50Q | D52Q |
| Q178D | Q176D |
| S87R | S88R |

In Tables 1 and 2 above, where the polypeptide having serine protease activity is defined by reference to mutation(s) in SEQ ID NO:2 (i.e. UniProt P16049-1), it will be appreciated that the mature protease will commence with 120 as its N-terminal amino acid. However, it will typically be expressed as a zymogen polypeptide having an activation sequence at its N-terminus (see above).

It will be appreciated by persons skilled in the art that the above identified mutations (defined by reference to the amino acid sequence of trypsin I of Atlantic cod, SEQ ID NO:1) could also be made in trypsins from other species. For example, the specific mutations highlighted in SEQ ID NOS: 3 and 4 (H25N and L160I, respectively with reference to SEQ ID NO:1 and PDB 2EEK!) could be made in the trypsin from Alaskan Pollock (for example see GenBank: BAH70476.3, wherein the amino acid sequence of the active trypsin commences at position 120, such that H25 corresponds to H29 in BAH70476.3, etc).

In an alternative embodiment, the polypeptide having serine protease activity comprises or consists of the amino acid sequence of a naturally-occurring serine protease. Thus, the polypeptide having serine protease activity may consist of the amino acid sequence of a naturally-occurring trypsin, of either eukaryotic or prokaryotic origin. Specifically included are cold-adapted trypsins, such as a trypsin from Atlantic cod (Gadus morhua), Atlantic and Pacific salmon (e.g. Salmo salar and species of Oncorhynchus) and Alaskan Pollock (Theragra chalcogramma). For example, the polypeptide having serine protease activity may comprise or consist of the amino acid of SEQ ID NO:1.

However, it will be appreciated by persons skilled in the art that such naturally-occurring serine protease polypeptides of the invention must be provided in a form different to that in which they are found in nature. For example, the polypeptide of the invention may consist of the amino acid sequence of a naturally-occurring eukaryotic trypsin but lack the glycosylation moieties present on the protein as it is expressed in nature.

It will also be appreciated by persons skilled in the art that the polypeptide having serine protease activity may also comprise or consist of a fragment of any of the above defined amino acid sequences, wherein the fragment exhibits an antimicrobial activity (as discussed above in relation to the first aspect of the invention).

Advantageously, the recombinant polypeptides of the second aspect of the invention exhibit one or more improved or otherwise beneficial properties relative to naturally-occurring serine proteases.

Thus, in one embodiment, the polypeptide exhibits improved stability relative to the trypsin I isolated from Atlantic cod (i.e. purified from cod and having the amino acid sequence of SEQ ID NO: 1 (PDB 2EEK!); which is commercially available as Penzyme® from Zymetech Ltd; see also EP 1 202 743 B, the relevant disclosures of which are incorporated herein by reference).

For example, the polypeptide having serine protease activity may exhibit improved thermal stability relative to the trypsin polypeptide of trypsin I isolated from Atlantic cod. By “thermal stability” we mean the ability of the polypeptide to retain it serine protease activity when exposed to high temperatures. Thermal stability may be assessed by determining the retention of serine protease activity when the polypeptide is stored at 60° C. for 3.5 hours (see Examples below).

Exemplary polypeptides of the invention with improved thermal stability include those polypeptides comprising or consisting of the amino acid of SEQ ID NO:1 (PDB 2EEK!), with one of the following defined mutations (see Table 1):
(a) K154E (“EZA-006”);
(b) N98T (“EZA-007”);
(c) I99L (“EZA-008”);
(d) V212I (“EZA-0010”);
(e) Y217D, M175K (“EZA-011”);
(f) Y217H (“EZA-012”);
(g) A229V (“EZA-014”);
(h) H25Y (“EZA-015”);
(i) H25N (“EZA-016”);
(j) H72N (“EZA-019”);
(k) R74E (“EZA-021”);
(l) N76L, Y82F (“EZA-023”);
(m) T79N (“EZA-025”);
(n) K49E, D50Q (“EZA-026”);
(o) S87R (“EZA-027”);
(p) E21T, H71D, D150S, K154V (“EZA-028”);
(q) 8179N, V233N (“EZA-029”);
(r) M135Q (“EZA-030”);
(s) M145K, V148G (“EZA-031”);
(t) L63I, S85A (“EZA-033”);
(u) L160I (“EZA-034”);
(v) V138I, L160A, A183V (“EZA-035”);
(w) V121I (“EZA-036”);
(x) V47I, V238I, M242I (“EZA-037”); and
(y) L234Y (“EZA-039”)
   wherein the mutation positions are identified with reference to the amino acid numbering in PDB entry no. 2EEK!.

Alternatively, or in addition, the polypeptide of the invention may exhibit improved autoproteolytic stability relative to the trypsin I isolated from Atlantic cod.

By “autoproteolytic stability” we mean the ability of the polypeptide to retain it serine protease activity without deactivation arising due to the polypeptide catalysing proteolytic cleavage of itself. Autoproteolytic stability may be assessed by determining the retention of serine protease activity when the polypeptide is stored at 25° C. for 8 hours (see Examples below).

Exemplary polypeptides of the invention with improved autoproteolytic stability include those polypeptides comprising or consisting of the amino acid of SEQ ID NO:1 (PDB 2EEK!) with one of the following defined mutations (see Table 1):
(a) I99L (“EZA-008”);
(b) V212I (“EZA-0010”);
(c) Y217D, M175K (“EZA-011”);
(d) Y217H (“EZA-012”);
(e) A229V (“EZA-014”);
(f) H25Y (“EZA-015”);
(g) H25N (“EZA-016”);
(h) H29Y (“EZA-017”);
(i) H72N (“EZA-019”);
(j) R74E (“EZA-021”);
(k) N76T (“EZA-022”);
(l) N76L, Y82F (“EZA-023”);
(m) T79S (“EZA-0024”);
(n) T79N (“EZA-025”);
(o) K49E, D50Q (“EZA-026”);
(p) S87R (“EZA-027”);
(q) E21T, H71D, D150S, K154V (“EZA-028”);
(r) S179N, V233N (“EZA-029”);
(s) M135Q (“EZA-030”);
(t) M145K, V148G (“EZA-031”);
(u) L63I, S85A (“EZA-033”);
(v) L160| (“EZA-034”);
(w) V138I, L160A, A183V (“EZA-035”);
(x) V121I (“EZA-036”); and
(y) V47I, V238I, M242I (“EZA-037”)
   wherein the mutation positions are identified with reference to the amino acid numbering in PDB entry no. 2EEK!.

It will be appreciated by persons skilled in the art that the polypeptides of the invention may exhibit improved catalytic activity relative to the trypsin I isolated from Atlantic cod (i.e. purified from cod and having the amino acid sequence of SEQ ID NO: 1; commercially available as Penzyme®).

For example, the polypeptide having serine protease activity may exhibit an improved (i.e. elevated) Kcat relative to trypsin I isolated from Atlantic cod.

Exemplary polypeptides of the invention with an improved Kcat include those polypeptides comprising or consisting of the amino acid of SEQ ID NO:1 (PDB 2EEK!) with one of the following defined mutations (see Table 1):
(a) H25N (“EZA-016”);
(b) H29Y (“EZA-017”);
(c) H71D (“EZA-018”);
(d) H72N (“EZA-019”);
(e) N76T (“EZA-022”);
(f) N76L, Y82F (“EZA-023”);
(g) M145K, V148G (“EZA-031”);
(h) M175Q (“EZA-032”);
(i) L63I, S85A (“EZA-033”);
(j) L160I (“EZA-034”);
(k) V138I, L160A, A183V (“EZA-035”);
(l) V47I, V238I, M242I (“EZA-037”); and
(m) V238I (“EZA-038”) wherein the mutation positions are identified with reference to the amino acid numbering in PDB entry no. 2EEK!.

Alternatively, or in addition, the polypeptide having serine protease activity may exhibit an improved (i.e. lower) Km relative to trypsin I isolated from Atlantic cod.

Exemplary polypeptides of the invention with an improved Km include those polypeptides comprising or consisting of the amino acid of SEQ ID NO:1 (PDB 2EEK!) with one of the following defined mutations (see Table 1):
(a) K154T (“EZA-003”);
(b) I99L (“EZA-008”);
(c) V212I (“EZA-0010”);
(d) Y217D, M175K (“EZA-011”);
(e) Y217S (“EZA-013”);
(f) A229V (“EZA-014”);
(g) H25Y (“EZA-015”);
(h) K49E, D50Q (“EZA-026”);
(i) E21T, H71D, D150S, K154V (“EZA-028”); and
(j) M135Q (“EZA-030”)
   wherein the mutation positions are identified with reference to the amino acid numbering in PDB entry no. 2EEK!.

Finally, the polypeptide having serine protease activity may exhibit an improved (i.e. elevated) specificity constant (Kcat/Km) relative to trypsin I isolated from Atlantic cod.

Exemplary polypeptides of the invention with an improved Kcat/Km include those polypeptides comprising or consisting of the amino acid of SEQ ID NO:1 (PDB 2EEK!) with one of the following defined mutations (see Table 1):
(a) K154T (“EZA-003”);
(b) Y217D, M175K (“EZA-011”);
(c) Y217S (“EZA-013”);
(d) A229V (“EZA-014”); and
(e) M135Q (“EZA-030”);
   wherein the mutation positions are identified with reference to the amino acid numbering in PDB entry no. 2EEK!.

It will be appreciated by persons skilled in the art that the polypeptides of the invention may undergo post-translation modification by the host cells. For example, where glycosylation of the recombinantly expressed polypeptide is desirable, yeast host cells may be used (such as Pichia pastoris).

However, bacterial host cells (such as Escherichia coli) do not permit glycosylation of the polypeptide. Hence, in one embodiment, the polypeptide of the invention is Don-glycosylated.

The term “amino acid” as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids. N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptides as defined herein comprise or consist of L-amino acids.

It will be appreciated by persons skilled in the art that the polypeptides of the invention may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. The term ‘peptidomimetic’ refers to a compound that mimics the conformation and desirable features of a particular peptide as a therapeutic agent.

For example, the said polypeptide includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will also be appreciated that the said polypeptide may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

It will be appreciated by persons skilled in the art that the present invention also includes pharmaceutically acceptable acid or base addition salts of the above described polypeptides. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

It will be further appreciated that the polypeptides of the invention may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

A third aspect of the invention provides an isolated nucleic acid molecule encoding an polypeptide having serine protease activity according to the second aspect of the invention. By “nucleic acid molecule” we include DNA (e.g. genomic DNA or complementary DNA) and mRNA molecules, which may be single- or double-stranded.

By “isolated” we mean that the nucleic acid molecule is not located or otherwise provided within a cell. In one embodiment, the nucleic acid molecule is a cDNA molecule.

Also included within the scope of the invention are the following:
(a) a fourth aspect of the invention provides a vector (such as an expression vector) comprising a nucleic acid molecule according to the third aspect of the invention; and
(b) a fifth aspect of the invention provides a host cell (such as a microbial or mammalian cell) comprising a nucleic acid molecule according to the third aspect of the invention or a vector according to the fourth aspect of the invention.

A sixth aspect of the invention provides a therapeutic composition comprising a pharmaceutically effective amount of a polypeptide according to the second aspect of the invention and a pharmaceutically-acceptable diluent, carrier, adjuvant or excipient.

Additional compounds may be included in the compositions, including, chelating agents such as EDTA, citrate, EGTA or glutathione. The antimicrobial/therapeutic compositions may be prepared in a manner known in the art that is sufficiently storage stable and suitable for administration to humans and animals. The therapeutic compositions may be lyophilised, e.g., through freeze drying, spray drying, spray cooling, or through use of particle formation from supercritical particle formation.

By “pharmaceutically acceptable” we mean a non-toxic material that does not decrease the effectiveness of the trypsin activity of the polypeptide of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), he disclosures of which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the therapeutic preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the polypeptide of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

In one embodiment, the polypeptide may be provided together with a stabiliser, such as calcium chloride.

The polypeptides of the invention may be formulated into any type of therapeutic composition known in the art to be suitable for the delivery of polypeptide agents.

In one embodiment, the polypeptides may simply be dissolved in water, saline, polyethylene glycol, propylene glycol, ethanol or oils (such as safflower oil, corn oil, peanut oil, cottonseed oil or sesame oil), tragacanth gum, and/or various buffers. For example, where the polypeptide is formulated to oral administration (such as in a mouth spray), the therapeutic composition may comprise the polypeptide dissolved in water, glycerol and menthol. An exemplary mouth spray formulation is marketed within Scandinavia as ColdZyme® (by Enzymatica AB, Lund, Sweden).

In a preferred embodiment, the invention provides a protease polypeptide as described above in an osmotically active solution. For example, the polypeptide may be formulated in glycerol or glycerine. Without wishing to be bound by theory, it is believed that such osmotically active solutions facilitate movement of fluid from within microbial cells to the extracellular milieu. This, in turn, is believed to facilitate the therapeutic effect of the polypeptides of the invention by creating a thin, active barrier that inhibits (at least, in part) the uptake of microbial cells such as bacteria and viruses by the host epithelial cells, e.g. of the oropharynx.

In a further embodiment, the therapeutic compositions of the invention may be in the form of a liposome, in which the polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, the disclosures of which are incorporated herein by reference.

The therapeutic compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the therapeutic compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

It will be appreciated that the therapeutic compositions of the invention may include ions and a defined pH for potentiation of action of the polypeptides. Additionally, the compositions may be subjected to conventional therapeutic operations such as sterilisation and/or may contain conventional adjuvants such as preservatives, stabilisers, wetting agents, emulsifiers, buffers, fillers, etc.

In one preferred embodiment, the therapeutic composition comprises the polypeptide in a Tris or phosphate buffer, together with one or more of EDTA, xylitol, sorbitol, propylene glycol and glycerol.

The therapeutic compositions according to the invention may be administered via any suitable route known to those skilled in the art. Thus, possible routes of administration include oral, buccal, parenteral (intravenous, subcutaneous, intratechal and intramuscular), topical, ocular, nasal, pulmonar, parenteral, vaginal and rectal. Also administration from implants is possible.

In an alternative embodiment, the therapeutic compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Alternatively, the therapeutic compositions may be administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the active polypeptide, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Advantageously, the polypeptide is provided in a form suitable for delivery to the mucosa of the mouth and/or oropharynx. For example, the polypeptide may be provided in a mouth spray, lozenge, pastille, chewing gum or liquid.

The therapeutic compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period.

The polypeptides can be formulated at various concentrations, depending on the efficacy/toxicity of the compound being used. Preferably, the formulation comprises the active agent at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM and most preferably about 500 μM.

Thus, the therapeutic formulation may comprise an amount of a polypeptide sufficient to kill or slow the growth of microorganisms, such as viruses, bacteria and yeasts, following administration to a subject.

A seventh aspect of the invention provides polypeptides having serine protease activity according to the second of the invention for use in medicine.

An eighth, related aspect of the invention provides a polypeptide as defined above in the preparation of a medicament for the treatment or prevention of a disorder or condition selected from the groups consisting of microbial infections, inflammation and wounds.

By “microbial infections” we include bacterial infections, viral infections, fungal infections, parasitic infections and yeast infections.

For example, the polypeptides of the invention may be for use in the treatment or prevention of a disorder or condition associated with a bacterial infection (with or without biofilm formation), such as periodontal disease.

Alternatively, the polypeptides of the invention may be for use in the treatment or prevention of a disorder or condition associated with a viral infection, such as the common cold and influenza. For example, the viral infection may be caused by an enterovirus (such as a human rhinovirus or Coxsackie A virus) or by a herpes simplex virus.

Additionally, the polypeptides of the invention may be for use in the treatment or prevention of a disorder or condition associated with a fungal infection, such as tinea pedis (athlete's foot) and candidiasis (thrush).

The polypeptides of the invention are particularly suitable for sue in a subject who has or is susceptible to an immunodeficiency.

By “immunodeficiency” we mean a condition in which the subject's immune disease is compromised, in whole or in part. The immunodeficiency may be acquired or secondary, e.g. following treatment with an immunosuppressive therapy, or may be primary, e.g. a naturally-occurring disorder in which part of the body's immune system is missing or does not function normally. Thus, in one embodiment the immunodeficiency is a secondary or acquired immunodeficiency.

For example, the immunodeficiency in the subject may arise from receiving treatment with an immunosuppressant therapy (such as glucocorticoids, cytostatics, antibodies, drugs acting on immunophilins, interferons, opioids, TNF-binding proteins, mycophenolate and radiation therapy).

Immunosuppressant therapies are commonly-used in medicine, for example:
(a) to prevent the rejection of transplanted organs and tissues (e.g. bone marrow, heart, kidney, liver);
(b) to treat autoimmune diseases or diseases that are of autoimmune origin (e.g. rheumatoid arthritis, multiple sclerosis, myasthenia gravis, systemic lupus erythematosus, sarcoidosis, focal segmental glomerulosclerosis, Crohn's disease, Behcet's Disease, pemphigus, and ulcerative colitis); and
(c) to treat other non-autoimmune inflammatory diseases (e.g. long term allergic asthma control).

In a further embodiment, the immunodeficiency is a naturally-occurring immunodeficiency. For example, the immunodeficiency may be due to a primary immunodeficiency (see below), a cancer (such as leukemia, lymphoma, multiple myeloma), chronic infection (such as acquired immunodeficiency syndrome or AIDS), malnutrition and/or aging.

Primary immunodeficiencies include a variety of disorders that render patients more susceptible to infections. If left untreated, these infections may be fatal. Common primary immunodeficiencies include disorders of humoral immunity (affecting B-cell differentiation or antibody production), T-cell defects and combined B- and T-cell defects, phagocytic disorders, and complement deficiencies. Major indications of these disorders include multiple infections despite aggressive treatment, infections with unusual or opportunistic organisms, failure to thrive or poor growth, and a positive family history. Early recognition and diagnosis can alter the course of primary immunodeficiencies significantly and have a positive effect on patient outcome.

Thus, the polypeptides of the invention are for use in the treatment or prevention of secondary infections of the mouth and/or oropharynx. For example, the polypeptides may be used in the treatment or prevention of rhinorrhea and/or fungal infection of the oral cavity and/or gum sores.

The polypeptides of the invention are particularly useful in the treatment or prevention of microbial infections in PI patients who suffer from regular episodes of infection (for example, at least five microbial infections a year, e.g. at least ten, fifteen, twenty, thirty or more microbial infections a year).

The polypeptides of the invention are also particularly useful in the treatment or prevention of microbial infections in athletes, especially professional athletes or other high-performance athletes. Over-training and/or prolonged physical exertion in such individuals can lead to temporary impairment of immune function, which can last several hours to days, rendering them vulnerable to microbial infections during that period (especially colds and ‘flu).

Thus, in one embodiment the polypeptides of the invention are for use in the treatment or prevention of microbial infections in marathon runners. The polypeptides may be administered immediately before the marathon (e.g. for one or two or more days prior to the event), on the day of the event itself and/or immediately after the marathon (e.g. for one or two or three or four or five or six or seven or more days after the event).

In an alternative embodiment, the polypeptides of the invention may be for use in the treatment or prevention of a disorder or condition associated with inflammation.

For example, the inflammatory disorder or condition may be selected from the group consisting of pain, acute inflammation, chronic inflammation, arthritis, inflamed joints, bursitis, osteoarthritis, rheumatoid arthritis, juvenile rheumatoid arthritis, septic arthritis, fibromyalgia, systemic lupus erythematosus, phlebitis, tendinitis, rash, psoriasis, acne, eczema, facial seborrheic eczema, and eczema of the hands, face or neck.

In a further embodiment, the polypeptides of the invention may be for use in the treatment or prevention of a wound, such as acute traumas (including burns), topical ulcers, scars, keloids, boils and warts.

Thus, the polypeptides of the invention may be provided in the form of a wound care product (i.e. in combination with a wound car material).

By “wound care material” we include substantially non-toxic materials suitable for use in wound care, including alginates, amorphous hydrogels, sheet hydrogels, hydrofibres and mixtures thereof.

The wound care product may take a number of different forms, depending on the constituent materials used and the intended purpose of the product. Typically, however, the product is provided in the form of dry non-woven sheets, freeze-dried sheets, solid gel sheets, ribbons, ropes and viscous gels, which may be used in or as a bandage or dressing.

Prior to use, the wound care product should be sterile and packaged in a microorganism-impermeable container. For example, the wound care product may be stored in a tube or other suitable sterile applicator.

Typically, the wound care product is applied directly to the surface of the wound. Optionally, a secondary conventional dressing may be applied over the top of the wound care product. Furthermore, in some cases, a permeable anti-adherence dressing may be applied between the wound and the wound care product.

The polypeptide are particularly suitable for debridement (i.e. removing infected, dead or peeling skin from otherwise healthy skin) and/or removal of fibrin clots.

It will be appreciated by persons skilled in the art that the polypeptides having serine protease activity of the invention may be for use in combination with one or more additional active agents.

For example, the additional active agents are selected from the group consisting of antimicrobial agents (including antibiotics, antiviral agents and anti-fungal agents), anti-inflammatory agents (including steroids and non-steroidal anti-inflammatory agents) and antiseptic agents.

Alternatively, or in addition, the polypeptides having serine protease activity of the invention may be for use in combination with one or more additional enzymes, such as glycosidases.

A ninth aspect of the invention provides the use of a polypeptide according to the second aspect of the invention in the preparation of a medicament for use in the treatment or prevention of a disorder or condition selected from the groups consisting of microbial infections, inflammation and wounds (such as those detailed above in relation to the eighth aspect of the invention).

A tenth aspect of the invention provides a method for the treatment or prevention in a subject of a disorder or condition selected from the groups consisting of microbial infections, inflammation and wounds (such as those detailed above in relation to the eighth aspect of the invention), the method comprising administering an effective amount of a polypeptide according to the second aspect of the invention.

In addition to numerous medical applications, the polypeptides having serine protease activity of the invention also have utility in cosmetic applications.

Thus, an eleventh aspect of the invention provides the use of a polypeptide according to the second aspect of the invention as a cosmetic therapy in a subject.

A thirteenth aspect of the invention provides a method of cosmetic therapy in a subject comprising administering an effective amount of a polypeptide according to the second aspect of the invention to a subject.

In one embodiment of the above aspects, the cosmetic therapy provides one or more of the following effects to the subject:
(a) exfoliating of skin (removal of dead and/or peeling skin cells);
(b) protecting against the breakdown of collagen and elastin in skin;
(c) a comedolytic effect;
(d) reducing or preventing glabellar (frown) lines; and/or
(e) promoting hair growth.

The polypeptides having serine protease activity of the invention also have utility as industrial agents.

Thus, a fourteenth aspect of the invention provides the use of a polypeptide according to the second aspect of the invention as an industrial.

For example, the polypeptides may be used as one or more of the following:
(a) a textile treatment agent;
(b) a biocatalyst (e.g. in the organic synthesis of pharmaceuticals)
(c) a cleaning/hygiene agent (e.g. a detergent);
(d) an environmental bioremediation agent (e.g. to reduce contamination);
(e) a molecular biology agent; and
(f) a food product treatment agent (e.g. in dairy manufacturing).

Preferences and options for a given aspect, feature or parameter of the invention should, unless the context indicates otherwise, be regarded as having been disclosed in combination with any and all preferences and options for all other aspects, features and parameters of the invention. For example, in one embodiment the invention provides a mouth spray comprising a trypsin polypeptide having the amino acid sequence of SEQ ID NO:3 or 4 for use in the treatment of prevention of bacterial or viral infection.

The listing or discussion of an apparently prior-published document in this specification should not necessarily be taken as an acknowledgement that the document is part of the state of the art or is common general knowledge.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the above description and the accompanying drawings. It should be understood, however, that the above description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The following drawing forms part of the present specification and is included to further demonstrate certain aspects of the present invention. The invention may be better understood by reference to this in combination with the detailed description of specific embodiments presented herein.

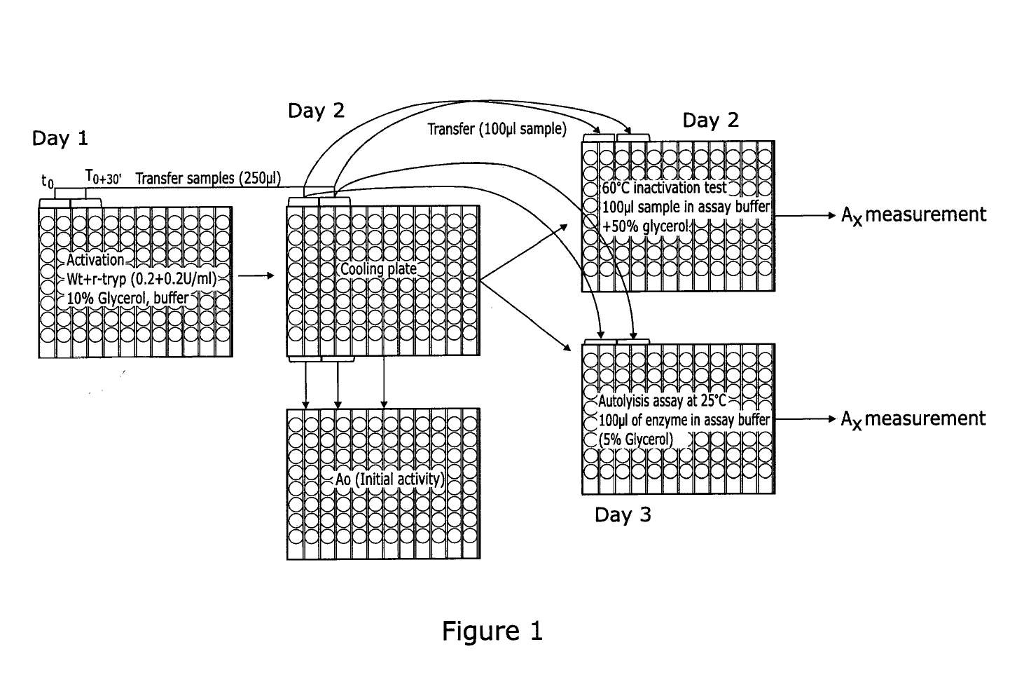

FIG. 1. Schematic description of the activation and stability test for recombinant trypsin.

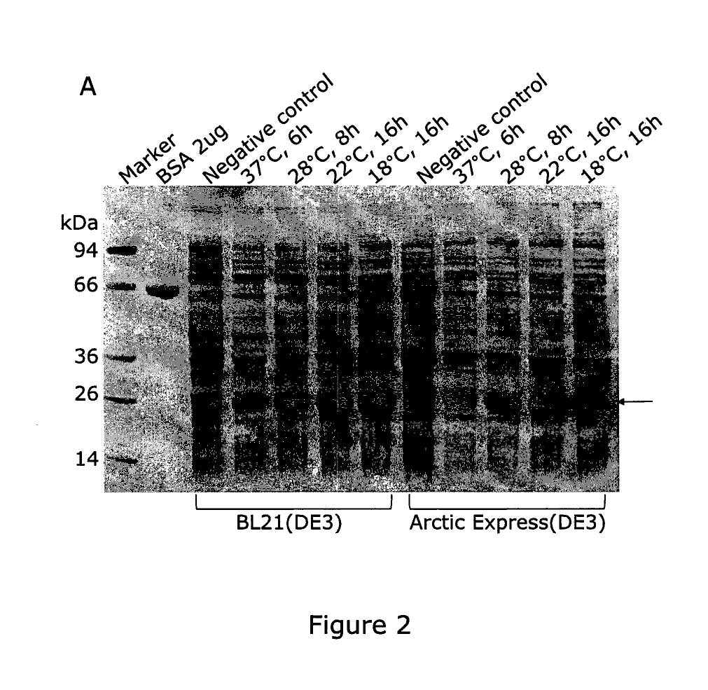

FIG. 2. Exemplary SDS-PAGE analysis of expression of EZA-034 in BL21(DE3) and ArcticExpress(DE3) host cells. Expression optimization of EZA-034 was performed in 4 ml LB medium with induction by 1 mM IPTG at four conditions 37° C. for 6 h, 28° C. for 8 h, 22° C. for 16 h or 18° C. for 16 h respectively.

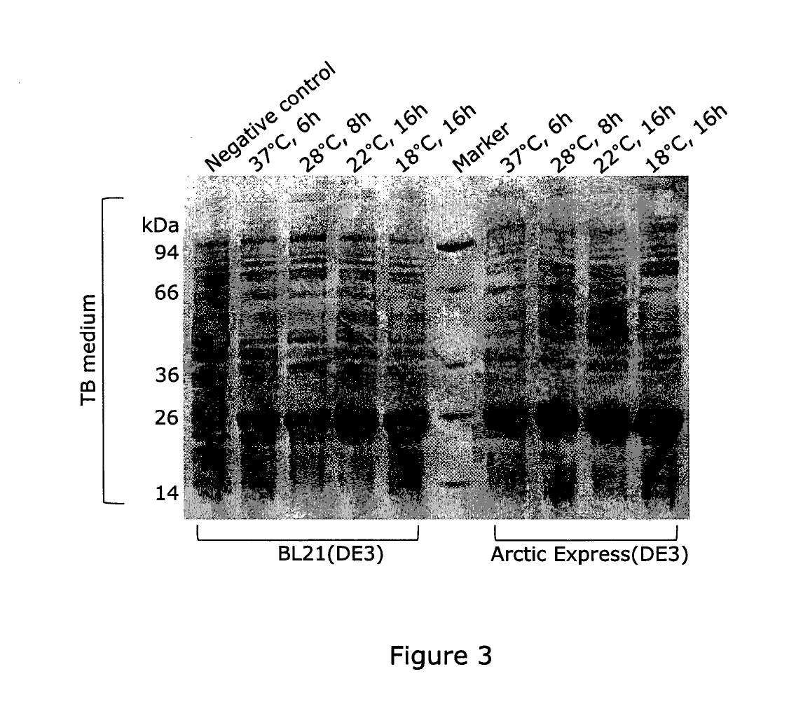

FIG. 3. Exemplary SDS-PAGE analysis of expression of EZA034. Expression optimization of EZA-034 was performed in 50 ml shake flasks using two strains BL21(DE3) and ArcticExpress(DE3), three mediums TB, TB plus 0.5% glucose and TB plus 0.5% glucose minus glycerol with induction by 1 mM IPTG at four conditions 37° C. for 6 h, 28° C. for 8 h, 22° C. for 16 h or 18° C. for 16 h respectively.

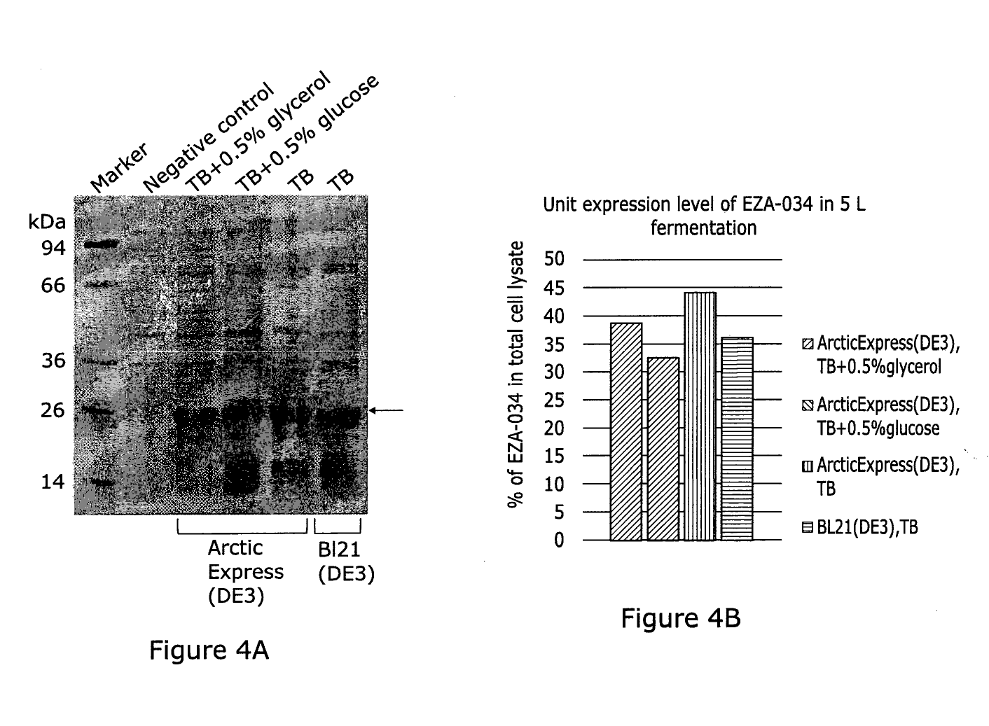

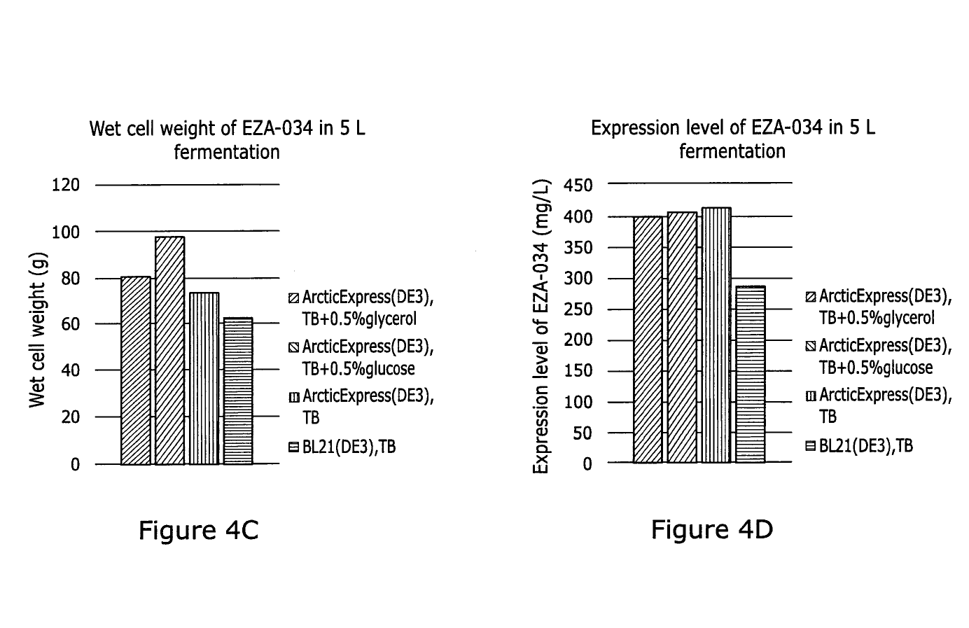

FIG. 4. Expression optimization of EZA-034 in 5 L fermentation in two E. coli strains BL21(DE3) and ArcticExpress(DE3), in three different media (TB+0.5% glycerol, TB+0.5% glucose and TB, with induction by 0.5 mM IPTG at 22° C. for 16 h). (A) SDS-PAGE analysis of expression of EZA-034, (B) Chart displays of the % of EZA-034 in total cell lysate, wherein the % was quantified by scanning the corresponding SDS-PAGE, (C) Chart displays of the wet cell weight of the tested conditions, and (D) Chart displays of the calculated expression level of EZA-034 in the tested conditions.

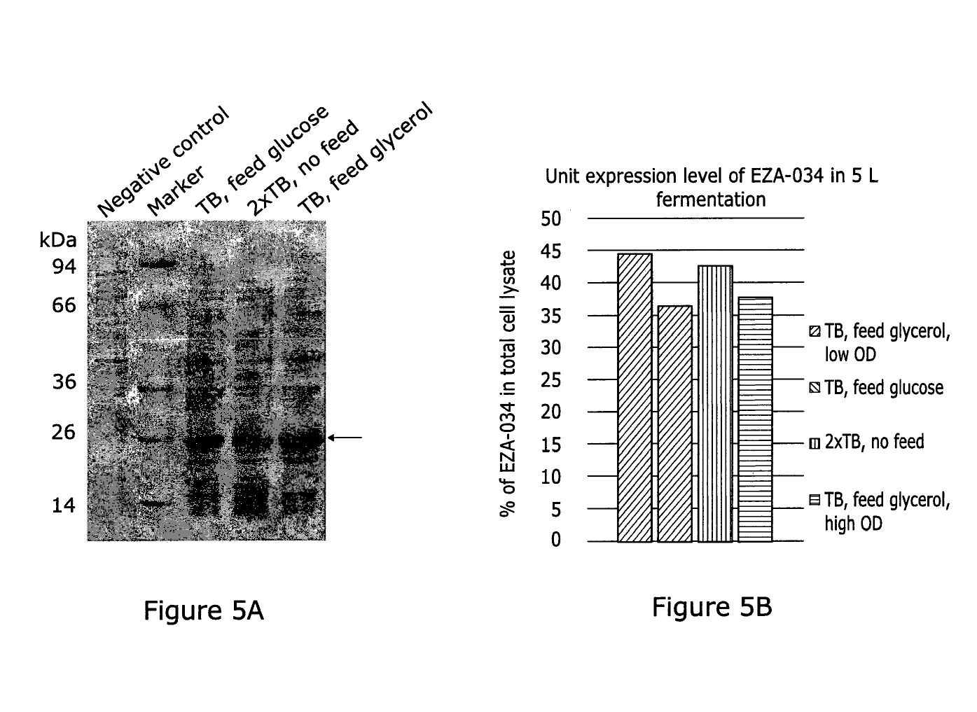

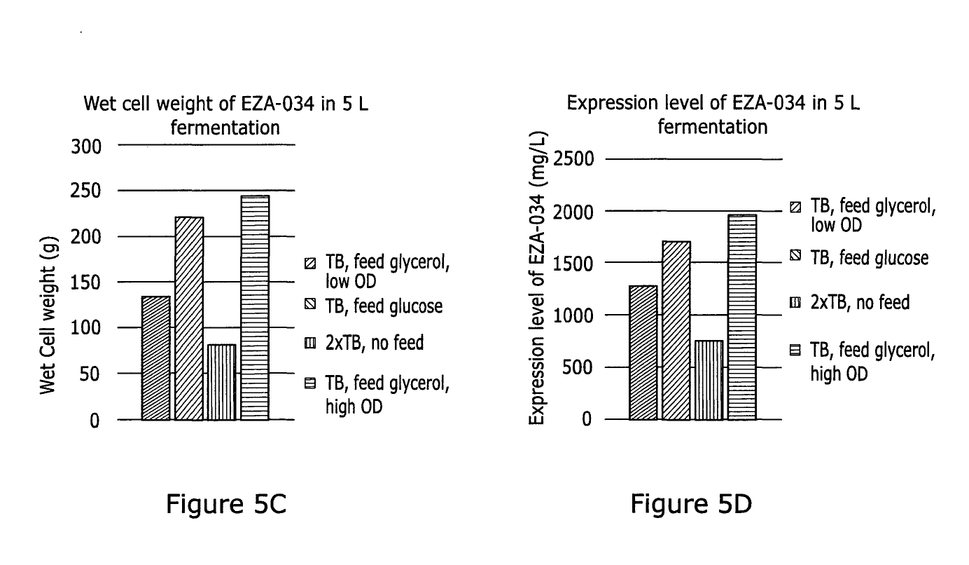

FIG. 5. Expression optimization of EZA-034 in 5 L fermentation in E. coli strain ArcticExpress(DE3), with induction by 0.5 mM IPTG at 22° C. for 16 h, in TB feed glycerol, TB feed glucose or 2×TB no feed respectively. (A) SDS-PAGE analysis of expression of EZA-034, (B) Chart displays the % of EZA-034 in total cell lysate, wherein the % was quantified by scanning the corresponding SDS-PAGE, (C) Chart displays the wet cell weight of the tested conditions, and (D) Chart displays the calculated expression level of EZA-034 in the tested conditions. The low or high OD refer to cell density at IPTG induction, the high OD of glycerol feed is equal to that of glucose feed.

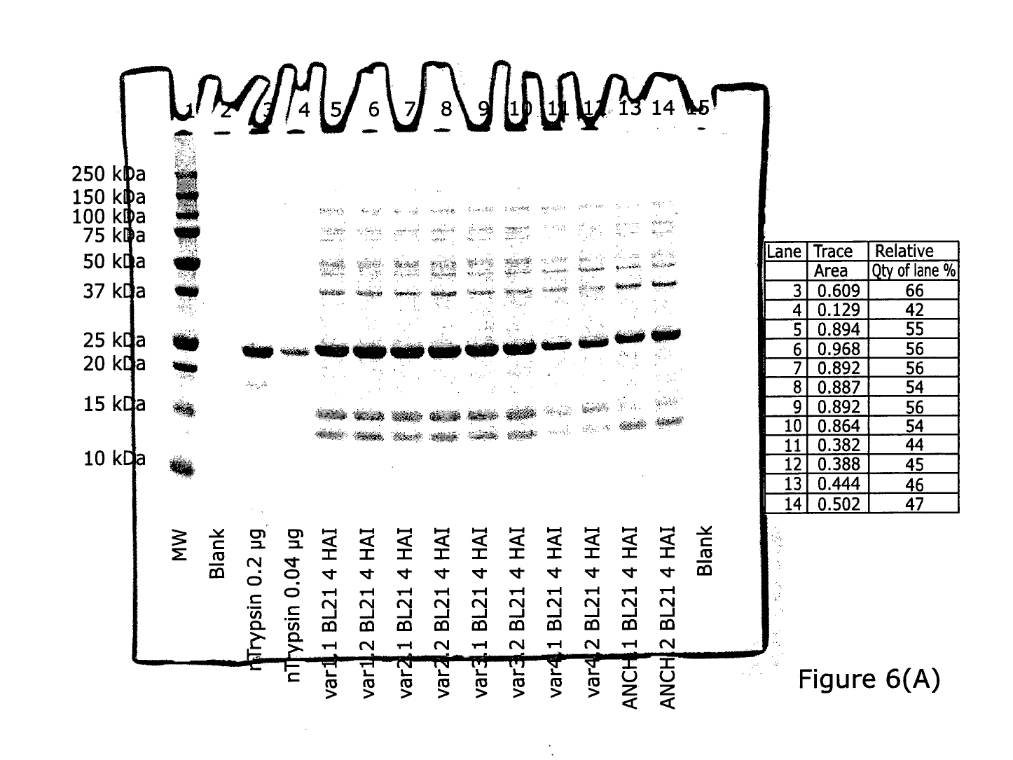

FIG. 6. SDS-PAGE analysis of recombinant expression of wildtype trypsin I from Atlantic cod, using four different activation peptides.

### EXAMPLES

### Example A—Production of Recombinant Serine Protease Polypeptides

### Cloning

A synthesized gene encoding the serine protease polypeptide of interest was cloned into E. coli expression E3 vector (GenScript) without any tag.

Nucleic acid encoding wildtype trypsin I from Atlantic cod is shown below in SEQ ID NO: 11 (in pUC57)

[SEQ ID NO: 11]

| | |
|---|---|
| 1 | GAAGAAGATA AAATCGTTGG CGGCTATGAA TGCACGAAAC |
| | ACTCGCAGGC ACACCAGGTC |
| 61 | TCACTGAACA GCGGTTACCA CTTTTGCGGC GGTAGTCTGG |
| | TTAGCAAAGA TTGGGTTGTT |
| 121 | AGTGCGGCCC ATTGCTATAA AAGCGTGCTG CGTGTTCGCC |
| | TGGGCGAACA TCACATTCGT |
| 181 | GTGAATGAAG GCACCGAACA GTACATTAGC TCTAGTAGCG |
| | TTATCCGCCA TCCGAACTAC |
| 241 | TCTAGTTACA ACATCAACAA CGATATCATG CTGATCAAAC |
| | TGACCAAACC GGCGACGCTG |
| 301 | AACCAGTATG TGCACGCCGT TGCACTGCCG ACCGAATGCG |
| | CAGCGGATGC AACCATGTGT |
| 361 | ACCGTGAGCG GCTGGGGTAA TACGATGAGC TCTGTTGCGG |
| | ATGGCGATAA ACTGCAGTGC |
| 421 | CTGTCTCTGC CGATTCTGAG TCATGCGGAT TGTGCCAACT |
| | CTTATCCGGG CATGATCACG |
| 481 | CAGAGCATGT TTTGCGCCGG TTACCTGGAA GGCGGTAAAG |
| | ATAGCTGCCA GGGTGATTCT |
| 541 | GGCGGTCCGG TGGTTTGTAA CGGCGTTCTG CAGGGTGTGG |
| | TTAGCTGGGG CTACGGTTGT |
| 601 | GCAGAACGTG ATCACCCGGG TGTCTATGCT AAAGTCTGTG |
| | TGCTGTCGGG CTGGGTCCGT |
| 661 | GATACGATGG CGAACTAT |

A number of nucleic acid molecules encoding mutated versions of trypsin I from Atlantic cod were synthesised by conventional techniques, i.e. directed mutagenesis by PCR.

### Refolding and Purification of Trypsin

Chemically competent E. coli BL21 (DE3) cells were transformed with the E3 vector containing the nucleotide sequence encoding the serine protease polypeptide (trypsin) of interest using standard procedures, i.e. heat shock transformation.

The zymogen polypeptide (trypsinogen) was overexpressed and formed inclusion bodies in the cytoplasm of the host cells.

The cells after induction were harvested and lysed by sonication. After centrifugation, inclusion bodies were washed in buffer (50 mM Tris, 10 mM EDTA, 2% Triton X-100, 300 mM NaCl, 2 mM DTT, pH8.0) and dissolved in 50 mM Tris, 8M Urea, pH8.0 and then dialyzed into 1×PBS, 10% Glycerol, pH7.4 at 4° C. overnight.

The purity of the expressed zymogen polypeptide from refolding exhibited >90% purity and no other purification was deemed necessary.

The recombinant zymogen polypeptide was then activated by adding wildtype trypsin I purified from Atlantic cod (0.2 U/ml) and incubating at room temperature for 24 hours (see Example B).

### Exemplary Polypeptides

The following polypeptides were obtained or produced:
(a) Wildtype trypsin I purified from Atlantic cod (“WT-Tryp” or “wildtype”);
(b) Recombinantly expressed wildtype trypsin I of Atlantic cod (SEQ ID NO:1, “R-Tryp”); and
(c) Thirty-eight different mutated versions of trypsin I of Atlantic cod (i.e. mutated sequences of SEQ ID NO:1).

The sequence mutations of the thirty-eight different mutated versions of cod trypsin I are shown in Table 1 (above).

The exemplary trypsin polypeptides were initially expressed as a zymogen polypeptide with the activation peptide MEEDK (SEQ ID NO: 5) fused to the N-terminus.

### Example B: Stability of Wildtype and Mutant Forms of Trypsin I of Atlantic Cod, Expressed Recombinantly

This example summarizes the results from the activation of 39 recombinant trypsin mutants expressed in E. coli. The activity of the recombinant trypsin polypeptides (R-Tryp) was activated by wildtype trypsin I purified from Atlantic cod (WT-Tryp) after a 24 hours incubation.

### Materials & Methods

### Expression of Recombinant Ttypsins

See Example A

### Assessment of Stability

The experiment designed for the activation and stability analysis of the recombinant samples was performed as follows (see FIG. 1):

### Day 1: Activation of Recombinant Trypsin

Recombinant enzymes (0.2 U/ml) were activated by wild type trypsin (0.2 U/ml) at room temperature during 24 hours in a microtiter plate. The samples were mixed with 20 mM Tris-HCl, 1 mM CaCl₂, 50% glycerol, pH 7.6 to a final volume of 200 μl.

### Day 2: Activity and Stability Measurements

The activated recombinant enzymes were transferred to a new microtiter plate (II) and kept on ice to keep the enzymes stable and stop the activation process.

### (a) Determination of Initial Activity A0

The activity of the activated enzyme (A0) was determined in a new microtiter plate (III) by mixing 245 μl of Gly-Pro-Arg in assay buffer, with 5 μl of recombinant enzyme from microtiter plate (II). The absorbance at 410 nm was followed and the activity was calculated according to the following formula:

$$U \text{/} ml = μmol \text{/} L \cdot s = \frac{{Slope}_{410 n m}^{*}}{{ɛ}^{*} {I}^{*}} {df}^{*} {60}^{*} {10}^{3} \qquad ( 1 )$$

where slope is the slope of the linear regression from the kinetic measurement of the trypsin activity at 30° C. during 200 seconds; df is the dilution factor, 60 is the conversion of seconds to minutes, c is the extension coefficient equal to 8800 M⁻¹ cm⁻¹, I is the length of the light path equal to 0.7109 cm, 10³ is the conversion mol/l to μmol/ml.

### (b) Temperature Inactivation

100 μl of the activated enzyme was transferred from microtiter plate (II) to a new microtiter plate (IV) and diluted to 200 μl to a final concentration of 50% glycerol, pH 7.6. Plate (IV) was incubated at 60° C. for 3.5 hours (WT-Tryp loses 90% of the initial activity). The remaining activity was determined as under (a).

### Day 3: Autocatalysis

100 μl of the activated enzyme was transferred from microtiter plate (II) to a new microtiter plate (V) containing 100 μl of 0.1 U/ml trypsin in 25% glycerol and assay buffer, pH 7.6. The plate was incubated at 25° C. for 8 hours (WT-Tryp loses 90% of the initial activity). The activity (A_{A}X) was determined as described under (a).

### Results

Activity, thermostability and autocatalysis of thirty-nine exemplary serine protease polypeptides is reported in table 3 (recombinant wildtype cod trypsin, EZA-001, and thirty-eight mutants thereof). There is a considerable difference in activity among the mutants. Several mutants expressed improved temperature stability in comparison to wildtype trypsin that only had 5% remaining activity and several mutants showed substantially improved autocatalytic stabilities in comparison to wildtype trypsin.

TABLE 3
Activity of 39 exemplary serine protease polypeptides

| | | Thermo- | Autocatalytic | | |
|---|---|---|---|---|---|
| | | stability: | stability: | | |
| | | Remaining | Remaining | Rela- | Relative |
| | Initial | activity after | activity after | tive | autoca- |
| | activity | inactivation | inactivation | thermo- | talytic |
| Sample | A0 | at 60° C., Ax | at 25° C., Ac | stability | stability |
| ID | (U/mg) | (U/ml) | (U/ml) | (Ax/A0) | (Ac/A0) |
| EZA001 | 0.52 | 0.10 | 0.07 | 0.20 | 0.13 |
| EZA002 | 0.48 | 0.05 | 0.01 | 0.11 | 0.02 |
| EZA003 | 0.52 | 0.09 | 0.05 | 0.18 | 0.09 |
| EZA004 | 0.48 | 0.09 | 0.04 | 0.19 | 0.07 |
| EZA005 | 0.36 | 0.07 | 0.02 | 0.19 | 0.06 |
| EZA006 | 0.39 | 0.10 | 0.03 | 0.26 | 0.07 |
| EZA007 | 0.30 | 0.10 | 0.01 | 0.33 | 0.04 |
| EZA008 | 0.36 | 0.11 | 0.09 | 0.31 | 0.26 |
| EZA009 | 0.35 | 0.06 | 0.03 | 0.16 | 0.09 |
| EZA010 | 0.44 | 0.12 | 0.12 | 0.28 | 0.28 |
| EZA011 | 0.36 | 0.10 | 0.11 | 0.28 | 0.31 |
| EZA012 | 0.35 | 0.13 | 0.11 | 0.37 | 0.31 |
| EZA013 | 0.36 | 0.07 | 0.05 | 0.20 | 0.13 |
| EZA014 | 0.41 | 0.10 | 0.07 | 0.25 | 0.17 |
| EZA015 | 0.39 | 0.09 | 0.11 | 0.24 | 0.27 |
| EZA016 | 0.36 | 0.22 | 0.21 | 0.60 | 0.56 |
| EZA017 | 0.35 | 0.14 | 0.15 | 0.41 | 0.42 |
| EZA018 | 0.39 | 0.05 | 0.02 | 0.13 | 0.04 |
| EZA019 | 0.37 | 0.10 | 0.10 | 0.27 | 0.27 |
| EZA020 | 0.39 | 0.07 | 0.03 | 0.19 | 0.08 |
| EZA021 | 0.38 | 0.11 | 0.09 | 0.30 | 0.23 |
| EZA022 | 0.49 | 0.09 | 0.17 | 0.18 | 0.34 |
| EZA023 | 0.39 | 0.18 | 0.16 | 0.47 | 0.41 |
| EZA024 | 0.43 | 0.09 | 0.07 | 0.21 | 0.17 |
| EZA025 | 0.39 | 0.17 | 0.14 | 0.43 | 0.37 |
| EZA026 | 0.33 | 0.15 | 0.15 | 0.44 | 0.46 |
| EZA027 | 0.34 | 0.10 | 0.06 | 0.30 | 0.17 |
| EZA028 | 0.35 | 0.16 | 0.18 | 0.45 | 0.51 |
| EZA029 | 0.35 | 0.16 | 0.16 | 0.46 | 0.45 |
| EZA030 | 0.33 | 0.16 | 0.11 | 0.50 | 0.33 |
| EZA031 | 0.40 | 0.14 | 0.15 | 0.35 | 0.36 |
| EZA032 | 0.44 | 0.07 | 0.02 | 0.16 | 0.03 |
| EZA033 | 0.42 | 0.12 | 0.10 | 0.27 | 0.24 |
| EZA034 | 0.41 | 0.13 | 0.11 | 0.31 | 0.27 |
| EZA035 | 0.42 | 0.12 | 0.16 | 0.29 | 0.38 |
| EZA036 | 0.38 | 0.11 | 0.13 | 0.30 | 0.34 |
| EZA037 | 0.36 | 0.10 | 0.16 | 0.27 | 0.44 |
| EZA038 | 0.41 | 0.08 | 0.05 | 0.20 | 0.12 |
| EZA039 | 0.38 | 0.10 | 0.04 | 0.26 | 0.11 |
| Wildtype | 0.16 | 0.01 | 0.01 | 0.05 | 0.08 |

### Example C: Activity Measurement of Recombinant Mutated Forms of Cod Trypsin I

### Materials & Methods

### Expression of Recombinant Polypeptides

Polypeptides corresponding to the wildtype amino acid sequence of trypsin I from Atlantic cod and thirty-eight mutated versions thereof were produced using the methods described in Example A.

### Activation

Activation of recombinant enzymes (approximately 0.01 mg/ml) was achieved by adding wild type trypsin (0.2 U/ml) at room temperature and incubate for 24 hours. The mixture was made in 20 mM Tris-HCl, 1 mM CaCl2, 50% glycerol, pH 8.0 to a final volume of 200 μl.

### Activity Assay to Determine Kinetic Constants

The substrate (Gly-Pro-Arg) was used at concentrations 0.005-0.15 mM in assay buffer containing 1% DMSO. 245 μL of substrate solutions were pipetted into a 96-well plate. The reaction was started by adding 5 μL of the sample mixture (above) and monitored at 410 nm in a SpectraMax plate reader. Kinetic measurement was performed every minute of a continuous 15-min run.

### Results

The results are shown in Table 4 below.

TABLE 4

| Sample | Parameter | Value | Relative to WT-Trp |
|---|---|---|---|
| WT-Trp | Vmax (Kcat) | 0.05372 | 100 |
| (purified) | Km | 0.00125 | 100 |
| | Vmax/Km | 43.07 | 100 |
| EZA-001 | Vmax | 0.05309 | 99 |
| | Km | 0.00087 | 70 |
| | Vmax/Km | 61.10 | 142 |
| EZA-002 | Vmax | 0.05292 | 99 |
| | Km | 0.00110 | 88 |
| | Vmax/Km | 48.09 | 112 |
| EZA-003 | Vmax | 0.05162 | 96 |
| | Km | 0.00050 | 40 |
| | Vmax/Km | 104.07 | 242 |
| EZA-004 | Vmax | 0.04380 | 82 |
| | Km | 0.00123 | 99 |
| | Vmax/Km | 35.47 | 82 |
| EZA-005 | Vmax | 0.05162 | 96 |
| | Km | 0.00094 | 75 |
| | Vmax/Km | 54.95 | 128 |
| EZA-006 | Vmax | 0.05289 | 98 |
| | Km | 0.00095 | 76 |
| | Vmax/Km | 55.81 | 130 |
| EZA-007 | Vmax | 0.05313 | 99 |
| | Km | 0.00114 | 91 |
| | Vmax/Km | 46.72 | 108 |
| EZA-008 | Vmax | 0.05084 | 95 |
| | Km | 0.00083 | 67 |
| | Vmax/Km | 60.90 | 141 |
| EZA-009 | Vmax | 0.05287 | 98 |
| | Km | 0.00087 | 70 |
| | Vmax/Km | 60.98 | 142 |
| EZA-010 | Vmax | 0.05046 | 94 |
| | Km | 0.00085 | 68 |
| | Vmax/Km | 59.44 | 138 |
| EZA-011 | Vmax | 0.05045 | 94 |
| | Km | 0.00077 | 62 |
| | Vmax/Km | 65.55 | 152 |
| EZA-012 | Vmax | 0.04208 | 78 |
| | Km | 0.00101 | 81 |
| | Vmax/Km | 41.74302 | 97 |
| EZA-013 | Vmax | 0.05006 | 93 |
| | Km | 0.00068 | 55 |
| | Vmax/Km | 73.65 | 171 |
| EZA-014 | Vmax | 0.05177 | 96 |
| | Km | 0.00083 | 66 |
| | Vmax/Km | 62.64 | 145 |
| EZA-015 | Vmax | 0.05060 | 94 |
| | Km | 0.00085 | 68 |
| | Vmax/Km | 59.36 | 138 |
| EZA-016 | Vmax | 0.05378 | 100 |
| | Km | 0.00103 | 83 |
| | Vmax/Km | 51.99 | 121 |
| EZA-017 | Vmax | 0.05457 | 102 |
| | Km | 0.00104 | 83 |
| | Vmax/Km | 52.53124 | 122 |
| EZA-018 | Vmax | 0.05962 | 111 |
| | Km | 0.00198 | 159 |
| | Vmax/Km | 30.04 | 70 |
| EZA-019 | Vmax | 0.05408 | 101 |
| | Km | 0.00115 | 92 |
| | Vmax/Km | 47.21 | 110 |
| EZA-020 | Vmax | 0.04421 | 82 |
| | Km | 0.00095 | 76 |
| | Vmax/Km | 46.77 | 109 |
| EZA-021 | Vmax | 0.05309 | 99 |
| | Km | 0.00128 | 103 |
| | Vmax/Km | 41.41 | 96 |
| EZA-022 | Vmax | 0.05436 | 101 |
| | Km | 0.00119 | 95 |
| | Vmax/Km | 45.85 | 106 |
| EZA-023 | Vmax | 0.05470 | 102 |
| | Km | 0.00137 | 110 |
| | Vmax/Km | 40.06 | 93 |
| EZA-024 | Vmax | 0.05120 | 95 |
| | Km | 0.00098 | 78 |
| | Vmax/Km | 52.36 | 122 |
| EZA-025 | Vmax | 0.05145 | 96 |
| | Km | 0.00090 | 72 |
| | Vmax/Km | 57.43 | 133 |
| EZA-026 | Vmax | 0.05042 | 94 |
| | Km | 0.00084 | 68 |
| | Vmax/Km | 59.70 | 139 |
| EZA-027 | Vmax | 0.05195 | 97 |
| | Km | 0.00094 | 76 |
| | Vmax/Km | 55.01 | 128 |
| EZA-028 | Vmax | 0.04167 | 78 |
| | Km | 0.00076 | 61 |
| | Vmax/Km | 54.60 | 127 |
| EZA-029 | Vmax | 0.05058 | 94 |
| | Km | 0.00091 | 73 |
| | Vmax/Km | 55.40 | 129 |
| EZA-030 | Vmax | 0.05109 | 95 |
| | Km | 0.00080 | 65 |
| | Vmax/Km | 63.47 | 147 |
| EZA-031 | Vmax | 0.05174 | 96 |
| | Km | 0.00103 | 83 |
| | Vmax/Km | 50.07 | 116 |
| EZA-032 | Vmax | 0.06226 | 116 |
| | Km | 0.00246 | 197 |
| | Vmax/Km | 25.31 | 59 |
| EZA-033 | Vmax | 0.05942 | 111 |
| | Km | 0.00166 | 133 |
| | Vmax/Km | 35.80 | 83 |
| EZA-034 | Vmax | 0.05672 | 106 |
| | Km | 0.00144 | 116 |
| | Vmax/Km | 39.29 | 91 |
| EZA-035 | Vmax | 0.05807 | 108 |
| | Km | 0.00162 | 130 |
| | Vmax/Km | 35.79 | 83 |
| EZA-036 | Vmax | 0.04887 | 91 |
| | Km | 0.00210 | 168 |
| | Vmax/Km | 23.28 | 54 |
| EZA-037 | Vmax | 0.05754 | 107 |
| | Km | 0.00172 | 138 |
| | Vmax/Km | 33.54 | 78 |
| EZA-038 | Vmax | 0.05786 | 108 |
| | Km | 0.00157 | 126 |
| | Vmax/Km | 36.74 | 85 |

### Example D: Strain Selection and Shaking Flask Optimization of EZA-034 Expression in E. coli

### Strain Selection

### Methods

Expression optimization was conducted using exemplary mutant trypsin EZA-034 (see Table 1; SEQ ID NO:4), expressed as a zymogen polypeptide with the activation peptide MEEDK (SEQ ID NO: 5) fused to the N-terminus.

The encoding nucleic acid comprised the nucleotide sequence of SEQ ID NO:12:

| [SEQ ID NO: 12] | |
|---|---|
| ATGGAAGAAG ATAAAATCGT TGGCGGCTAT GAATGCACGA AACACTCGCA GGCACACCAG | 61 |
| GTCTCACTGA ACAGCGGTTA CCACTTTTGC GGCGGTAGTC TGGTTAGCAA AGATTGGGTT | 121 |
| GTTAGTGCGG CCCATTGCTA TAAAAGCGTG CTGCGTGTTC GCCTGGGCGA ACATCACATT | 181 |
| CGTGTGAATG AAGGCACCGA ACAGTACATT AGCTCTAGTA GCGTTATCCG CCATCCGAAC | 241 |
| TACTCTAGTT ACAACATCAA CAACGATATC ATGCTGATCA AACTGACCAA ACCGGCGACG | 301 |
| CTGAACCAGT ATGTGCACGC CGTTGCACTG CCGACCGAAT GCGCAGCGGA TGCAACCATG | 361 |
| TGTACCGTGA GCGGCTGGGG TAATACGATG AGCTCTGTTG CGGATGGCGA TAAACTGCAG | 421 |
| TGCCTGTCTA TTCCGATTCT GAGTCATGCG GATTGTGCCA ACTCTTATCC GGGCATGATC | 481 |
| ACGCAGAGCA TGTTTTGCGC CGGTTACCTG GAAGGCGGTA AAGATAGCTG CCAGGGTGAT | 541 |
| TCTGGCGGTC CGGTGGTTTG TAACGGCGTT CTGCAGGGTG TGGTTAGCTG GGGCTACGGT | 601 |
| TGTGCAGAAC GTGATCACCC GGGTGTCTAT GCTAAAGTCT GTGTGCTGTC GGGCTGGGTC | 661 |
| CGTGATACGA TGGCGAACTA TTAA | |

Experiments were performed in 4 ml LB medium using four E. coli strains BL21(DE3), ArcticExpress(DE3), and BL21Star(DE3), with induction by 1 mM IPTG at four conditions 37° C. for 6 h, 28° C. for 8 h, 22° C. for 16 h or 18° C. for 16 h respectively.

### Results & Conclusions

FIG. 2 shows an exemplary SDS-PAGE analysis of expression of EZA-034 in BL21(DE3) and ArcticExpress(DE3) host cells.

Table 5 below shows the % of EZA-034 in total cell lysate, quantified by scanning the corresponding SDS-PAGE.

TABLE 5

| | Host cell | | |
|---|---|---|---|
| Culture conditions | BL21(DE3) | ArcticExpress(DE3) | BL21Star(DE3) |
| 37 C., 6 h | 25.2 | 28.0 | 18.8 |
| 28 C., 8 h | 18.9 | 36.9 | 24.1 |
| 22 C., 16 h | 28.4 | 32.2 | 23.0 |
| 18 C., 16 h | 28.0 | 21.4 | 26.0 |

The strain ArcticExpress(DE3), with induction with 1 mM IPTG at 28° C. for 8 h, exhibited the highest unit expression (expression of target protein per cell) of EZA-034.

### Expression Optimization

### Methods

Expression optimization of EZA-034 was performed in 50 ml shake flasks using two strains BL21(DE3) and ArcticExpress(DE3).

Three mediums were studies: TB, TB plus 0.5% glucose and TB plus 0.5% glucose minus glycerol, with induction by 1 mM IPTG at one of four conditions (37° C. for 6 h, 28° C. for 8 h, 22° C. for 16 h or 18° C. for 16 h).

### Results & Conclusions

FIG. 3 shows an exemplary SDS-PAGE analysis of expression of EZA-034.

Table 6 below shows the % of EZA-034 in total cell lysate. The % was quantified by scanning the corresponding SDS-PAGE.

TABLE 6

Culture medium

| Host cell and | | | TB + glucose − |
|---|---|---|---|
| culture conditions | TB | TB + glucose | glycerol |
| BL21(DE3) | 29.3 | 19.6 | 20.0 |
| 37° C. for 6 h | | | |
| BL21(DE3 | 37.0 | 26.3 | 21.0 |
| 28° C. for 8 h | | | |
| BL21(DE3 | 32.1 | 27.9 | 32.3 |
| 22° C. for 16 h | | | |
| BL21(DE3 | 38.0 | 27.6 | 18.5 |
| 18° C. for 16 h | | | |
| ArcticExpress(DE3) | 32.8 | 19.0 | 26.0 |
| 37° C. for 6 h | | | |
| ArcticExpress(DE3) | 38.6 | 31.8 | 40.6 |
| 28° C. for 8 h | | | |
| ArcticExpress(DE3) | 34.9 | 30.4 | 31.7 |
| 22° C. for 16 h | | | |
| ArcticExpress(DE3) | 37.9 | 32.6 | 37.7 |
| 18° C. for 16 h | | | |

Table 7 below shows the wet cell weight of the tested conditions.

TABLE 7

| | Culture medium | | |
|---|---|---|---|
| Host cell and | | | TB + glucose − |
| culture conditions | TB | TB + glucose | glycerol |
| BL21(DE3) | 0.4 | 0.5 | 0.4 |
| 37° C. for 6 h | | | |
| BL21(DE3 | 0.6 | 0.5 | 0.7 |
| 28° C. for 8 h | | | |
| BL21(DE3 | 0.7 | 0.5 | 0.6 |
| 22° C. for 16 h | | | |
| BL21(DE3 | 0.9 | 0.7 | 0.8 |
| 18° C. for 16 h | | | |
| ArcticExpress(DE3) | 0.5 | 0.5 | 0.4 |
| 37° C. for 6 h | | | |
| ArcticExpress(DE3) | 0.6 | 0.5 | 0.5 |
| 28° C. for 8 h | | | |
| ArcticExpress(DE3) | 0.9 | 1.0 | 0.5 |
| 22° C. for 16 h | | | |
| ArcticExpress(DE3) | 0.7 | 0.5 | 0.5 |
| 18° C. for 16 h | | | |

Table 8 below shows the calculated expression level of EZA-034 in the tested conditions.

TABLE 8

Culture medium

| Host cell and | | | TB + glucose − |
|---|---|---|---|
| culture conditions | TB | TB + glucose | glycerol |
| BL21(DE3) | 149.5 | 124.7 | 102.2 |
| 37° C. for 6 h | | | |
| BL21(DE3 | 283.2 | 167.6 | 187.5 |
| 28° C. for 8 h | | | |
| BL21(DE3 | 286.8 | 178.3 | 247.1 |
| 22° C. for 16 h | | | |
| BL21(DE3 | 436.9 | 246.5 | 188.9 |
| 18° C. for 16 h | | | |
| ArcticExpress(DE3) | 209.2 | 120.9 | 132.5 |
| 37° C. for 6 h | | | |
| ArcticExpress(DE3) | 295.1 | 203.0 | 258.8 |
| 28° C. for 8 h | | | |
| ArcticExpress(DE3) | 400.7 | 388.2 | 202.5 |
| 22° C. for 16 h | | | |
| ArcticExpress(DE3) | 338.1 | 208.1 | 240.6 |
| 18° C. for 16 h | | | |

Strains ArcticExpress(DE3) with induction with 1 mM IPTG at 22° C., 16 h and BL21(DE3) with induction with 1 mM IPTG at 18° C., 16 h were found to exhibit the highest expression of EZA-034.

The best two mediums were TB and TB plus 0.5% glucose.

### Example E: Fermentation Optimization of EZA-034 in 5 L Scale

### Medium and Strain Selection

### Methods

Expression optimization of EZA-034 in 5 L fermentation was performed using two E. coli strains BL21(DE3) and ArcticExpress(DE3), in three different media (TB+0.5% glycerol, TB+0.5% glucose and TB, with induction by 0.5 mM IPTG at 22° C. for 16 h).

### Results & Conclusions

FIG. 4 shows (A) SDS-PAGE analysis of expression of EZA-034, (B) Chart displays of the % of EZA-034 in total cell lysate, wherein the % was quantified by scanning the corresponding SDS-PAGE, (C) Chart displays of the wet cell weight of the tested conditions, and (D) Chart displays of the calculated expression level of EZA-034 in the tested conditions.

The strain ArcticExpress(DE3), with induction with 0.5 mM IPTG at 22° C. for 16 h was chosen for feed culture fermentation for expression of EZA-034.

### Feed Culture Selection in 5 L Fermentation

### Methods

Expression optimization of EZA-034 in 5 L fermentation was performed using E. coli strain ArcticExpress(DE3), with induction by 0.5 mM IPTG at 22° C. for 16 h, in TB feed glycerol, TB feed glucose or 2×TB no feed respectively. The seed culture was 3%. The pH was controlled around 6.8 by adding 30% NH₄OH or adding the feed culture. Air flow was controlled from 3 L/min to 9 L/min according to the dissolved oxygen level.

FIG. 5 shows (A) SDS-PAGE analysis of expression of EZA-034, (B) Chart displays the % of EZA-034 in total cell lysate, wherein the % was quantified by scanning the corresponding SDS-PAGE, (C) Chart displays the wet cell weight of the tested conditions, and (D) Chart displays the calculated expression level of EZA-034 in the tested conditions.

The feed culture of glycerol resulted in the highest unit expression level, and when the OD at induction are same, final expression level of EZA-034 in glycerol feed is higher than that of glucose feed, about 1.9 g/L.

Reproducibility testing showed that the expression level of EZA-034 in E. coli ArcticExpress(DE3) stays constant after re-production for 9 times (data not shown). It is thus suitable for large-scale production and long term storage.

### Example F: Purification, Endotoxin Removal and Refolding of EZA-034

### Inclusion Body Washing

Cells were collected by centrifugation at 8,000 g, 4° C. for 20 min, and the wet pellets were weighed. Total 10 g/tube wet pellets were re-suspended and lyzed by sonication in lysis buffer at 50% full power for 3 sec, on ice 6 sec for a total of 30 min. The inclusion bodies were spun down at 13,000 rpm, 4° C. for 30 min and washed as follows:
Cell lysis: TND buffer (50 mM Tris-HCl, 150 mM NaCl, 10 mM DTT, PH 8.0) plus 1% Triton
Wash 1: IB wash buffer (100 mM Tris-HCl, 300 mM NaCl, 10 mM EDTA, 1% Triton X-100, 10 mM DTT, pH 8.0)
Wash 2: IB wash buffer (100 mM Tris-HCl, 300 mM NaCl, 10 mM EDTA, 1% Triton X-100, 10 mM DTT, pH 8.0)
Wash 3: IB wash buffer (100 mM Tris-HCl, 300 mM NaCl, 10 mM EDTA, 1% Triton X-100, 10 mM DTT, pH 8.0) plus 2 M urea
Wash 4: IB wash buffer (100 mM Tris-HCl, 300 mM NaCl, 10 mM EDTA, 1% Triton X-100, 10 mM DTT, pH 8.0) plus 2 M urea
Wash 5: TND buffer plus 4 M urea
Wash 6: TND buffer plus 4 M urea
Wash 7: TND buffer plus 4 M urea

In brief, inclusion bodies were re-suspended by stirring at 4° C. with wash buffers, homogenized by sonication at 50% full power for 3 sec, on ice 6 sec for a total of 10 min, and centrifuged at 13,000 rpm for 30 min at 4° C. Finally, the inclusion bodies were solubilized in 40 ml of 50 mM Tris-HCl, 8 M urea, 10 mM DTT, pH 8.0 and incubated for 30 min at room temperature. The sample was spun at 44,000 rpm for 30 min at 15° C. And the supernatant was used for further purification.

Purification with an Ion Exchange Column

Optimization of purification resin and volume of resin were carried out. 20 ml solubilized protein (9 mg/ml as determined by Bradford Assay) was purified by 1 ml SP Sepharose Fast Flow and the flow through was purified by 1 ml Q Sepharose Fast Flow. SDS-PAGE was used to analyze the purification process.

5 ml solubilized protein was purified by 1 ml Q Sepharose Fast Flow, and 5 ml was purified by 1 ml SP Sepharose Fast Flow, respectively. SDS-PAGE was used to analyze the purification process.

Ion exchange Q Sepharose was used to improve the purity. Target protein bound to the column and was eluted with 20 mM NaCl. The binding capacity of Q Sepharose was about 20 mg EZA-034 per milliliter.

### Scale-Up Purification of EZA-034

Total 45 g wet pellet (from one liter expression) was lyzed, and inclusion bodies were washed and solubilized using the optimized conditions. Total 100 ml solubilized protein (9.0 mg/ml) was purified with Q Sepharose Fast Flow column (volume about 50 ml) at flow rate of 3.5 ml/min with 50 mM Tris-HCl, 8 M Urea, 4 mM DTT, pH 8.0 (Non-pyrogenic) as running buffer, collected 1.8 ml fractions. SDS-PAGE is used to analyze the purification process.

### Refolding Optimisation of EZA-034

The purified EZA-034 (10.0 mg/ml, 95% purity, storage buffer was 50 mM Tris-HCl, 8 M Urea, 30 mM NaCl, 4 mM DTT, pH 8.0) was refolded in 20 mM Tris-HCl, 2 mM DTT, 1 mM CaCl2, 10% glycerol, pH 7.6, concentration of EZA-034 could reach 3 mg/ml.

Refolding of EZA-034 was also possible in 1×PBS, 10% glycerol, pH 7.4.

The endotoxin level of EZA-034 after refolding was between 5 and 10 EU/mg as determined by LAL method. The yield of EZA-034 after refolding was about 500 mg/L, purity was higher than 95% as determined by SDS-PAGE.

### Example G: Assessment of Alternative Activation Peptides

In the above examples, the polypeptide having serine protease activity is expressed as a zymogen polypeptide with the activation peptide MEEDK (SEQ ID NO: 5) fused to the N-terminus.

In this study, the effect of four different activation peptides was tested on expression and refolding in BL21(DE3) cells of wildtype trypsin1 of Atlantic cod (see SEQ ID NO:2 below).

```
[SEQ ID NO: 2]
                10 20 30 40
MKSLIFVLLL GAV [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

wherein:
Signal peptide=amino acids 1 to 13 (underlined)
Propeptide=amino acids 14 to 19 (bold italics)
Mature trypsin=amino acids 20 to 241

The four variant activation peptides tested to be expressed and tested were as follows:

### (a) Variant 1

```
[SEQ ID NO: 13]
                10 20 30 40
                          M [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

### (b) Variant 2

```
[SEQ ID NO: 14]
                10 20 30 40
                        MV [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

### (c) Variant 3

```
[SEQ ID NO: 15]
                10 20 30 40
                        MA [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

### (d) Variant 4

```
[SEQ ID NO: 16]
                10 20 30 40
                    MGAV [symbol] I VGGYECTKHS QAHQVSLNSG
                50 60 70 80
YHFCGGSLVS KDWVVSAAHC YKSVLRVRLG EHHIRVNEGT
                90 100 110 120
EQYISSSSVI RHPNYSSYNI NNDIMLIKLT KPATLNQYVH
              130 140 150 160
AVALPTECAA DATMCTVSGW GNTMSSVADG DKLQCLSLPI
              170 180 190 200
LSHADCANSY PGMITQSMFC AGYLEGGKDS CQGDSGGPVV
              210 220 230 240
CNGVLQGVVS WGYGCAERDH PGVYAKVCVL SGWVRDTMAN Y
```

The following two activation sequences were also used in this study as controls:

[SEQ ID NO: 18]
(i) [symbol] . . . (“nTrypsin”; i.e. SEQ ID NO: 2
within the signal sequence);
and
[SEQ ID NO: 17]
(ii) MRPLVFLVLLGAA [symbol] . . . (“ANCH”; sequence
derived from anchovy pre-trypsinogen)

wherein:
Signal peptide=underlined
Propeptide=bold italics
. . . =Mature trypsin sequence commencing with IVGG

Expression and refolding was assessed using SDS-PAGE, run in duplicate for each activation peptide (see FIG. 6).

### REFERENCES

1. Kuddus M, Ramteke P W. Recent developments in production and biotechnological applications of cold-active microbial proteases. Crit Rev Microbiol. 2012; 38:330-8.
2. Fein H, Maytin E V, Mutasim D F, Bailin P L. Topical protease therapy as a novel method of epidermal ablation: preliminary report. Dermatol Surg. 2005; 31:139-47.
3. Craik C S, Page M J, Madison E L. Proteases as therapeutics. Biochem J. 2011; 435:1-16.
4. Duffy M J, McGowan P M, Gallagher W M. Cancer invasion and metastasis: changing views. J Pathol. 2008; 214:283-93.
5. Scott C J, Taggart C C. Biologic protease inhibitors as novel therapeutic agents. Biochimie. 2010; 92:1681-8.
6. Rawlings N D, Tolle D P, Barrett A J. Evolutionary families of peptidase inhibitors. Biochem J. 2004; 378:705-16.
7. Morris R T. The action of trypsin, pancreatic extract and pepsin upon sloughs, coagula, and mucopus. NY Med J. 1891; 53:424-6.
8. Morani A D. Trypsin therapy in the management of chronic surface ulcers. Plast Reconstr Surg. 1953; 11:372-9.
9. Rapoport C. The use of trypsin in the therapy of tuberculous lymphadenitis and tuberculous fistulae. Dis Chest. 1958; 34:154-61.
10. Gudmundsdottir A, Palsdottir H M. Atlantic cod trypsins: from basic research to practical applications. Mar Biotechnol. 2005; 7:77-88.
11. Seiberg M, Siock P, Wisniewski S, Cauwenbergh G, Shapiro S S. The effects of trypsin on apoptosis, utriculi size, and skin elasticity in the Rhino mouse. J Investig Dermatol. 1997; 109:370-6.
12. Shi L, Carson D. Collagenase Santyl ointment: a selective agent for wound debridement. J Wound Ostomy Cont Nurs. 2009; 36(Suppl.):S12-6.
13. Hellgren L. Cleansing properties of stabilized trypsin and streptokinase-streptodornase in necrotic leg ulcers. Eur J Clin Pharmacol. 1983; 24:623-8.
14. Brooks J L, Jefferson K K. Staphylococcal biofilms: quest for the magic bullet. Adv Appl Microbiol. 2012; 81:63-87.
15. Chaignon P, Sadovskaya I, Ragunah C, Ramasubbu N, Kaplan J B, Jabbouri S. Susceptibility of staphylococcal biofilms to enzymatic treatments depends on their chemical composition. Appl Microbiol Biotechnol. 2007; 75:125-32.
16. Marti M, Trotonda M P, Tormo-Mas M A, et al. Extracellular proteases inhibit protein-dependent biofilm formation in Staphylococcus aureus. Microbes Infect. 2010; 12:55-64.
17. Hangler M, Burmolle M, Schneider I, Allermann K, Jensen B. The serine protease Esperase HPF inhibits the formation of multispecies biofilm. Biofouling. 2009; 25:667-74.
18. Siddiqui K S, Cavicchioli R. Cold-adapted enzymes. Ann Rev Biochem. 2006; 75:403-33.
19. Lonhienne T, Gerday C, Feller G. Psychrophilic enzymes: revisiting the thermodynamic parameters of activation may explain local flexibility. Biochim Biophys Acta. 2000; 1543:1-10.
20. Aghajari N, Van Petegem F, Villeret V, et al. Crystal structures of a psychrophilic metalloprotease reveal new insights into catalysis by cold-adapted proteases. Proteins. 2003; 50:636-47.
21. Gerday C, Aittaleb M, Bentahir M, et al. Cold-adapted enzymes: from fundamentals to biotechnology. Trends Biotechnol. 2000; 18:103-7.
22. Asgeirsson B, Fox J W, Bjarnason J B. Purification and characterization of trypsin from the poikilotherm Gadus morhua. Eur J Biochem. 1989; 180:85-94.
23. Osnes K K, Mohr V. On the purification and characterization of three anionic, serine-type peptide hydrolases from antarctic krill, Euphausia superba. Comp Biochem Physiol B. 1985; 82:607-19.
24. Stefansson B, Helgadottir L, Olafsdottir S, Gudmundsdottir A, Bjarnason J B. Characterization of cold-adapted Atlantic cod (Gadus morhua) trypsin I-kinetic parameters, autolysis and thermal stability. Comp Biochem Physiol B: Biochem Mol Biol. 2010; 155:186-94.
25. Leiros H K, Willassen N P, Smalas A O. Structural comparison of psychrophilic and mesophilic trypsins. Elucidating the molecular basis of cold-adaptation. Eur J Biochem. 2000; 267:1039-49.

1. A method for the production of a recombinant polypeptide having serine protease activity comprising
(a) transforming a microbial host cell, or population thereof, with a nucleic acid molecule encoding a zymogen polypeptide comprising an activation peptide fused to the N-terminus of a polypeptide having serine protease activity
wherein the zymogen polypeptide lacks a signal sequence;
(b) expressing said zymogen polypeptide in the host cell(s) as inclusion bodies;
(c) purifying the zymogen polypeptide from the host cell(s); and
(d) activating the zymogen polypeptide by exposure to a protease, such as a trypsin
wherein step (c) comprises solubilising the zymogen polypeptide from the inclusion bodies and refolding the polypeptide into a bioactive form.

2. A method according to claim 1 wherein the polypeptide having serine protease activity exhibits trypsin activity.

3. A method according to claim 1 or 2 wherein the polypeptide having serine protease activity comprises or consists of an amino acid sequence which shares at least 70% sequence identity with amino acid sequence of SEQ ID NO:1, for example at least 80%, 85%, 90%, 95%, 95%, 97%, 98% or 99% sequence identity
```
[SEQ ID NO: 1]
  16
I
IVGGYECTKHSQAHQVSLNSGYHFCGGSLVSKDWVVSAAHCYKSVLRVRL
GEHHIRVNEG
  79
I
TEQYISSSSVIRHPNYSSYNINNDIMLIKLTKPATLNQYVHAVALPTECA
ADATMCTVSG
141
I
WGNTMSSVADGDKLQCLSLPILSHADCANSYPGMITQSMFCAGYLEGGKD
SCQGDSGGPV
200
I
VCNGVLQGVVSWGYGCAERDHPGVYAKVCVLSGWVRDTMANY
```
or a fragment thereof which exhibits an antimicrobial activity.

4. A method according to claim 3 wherein the polypeptide having serine protease activity does not comprise histidine at position 25.

5. A method according to claim 4 wherein the polypeptide having serine protease activity comprises or consists of the amino acid sequence of SEQ ID NO:3 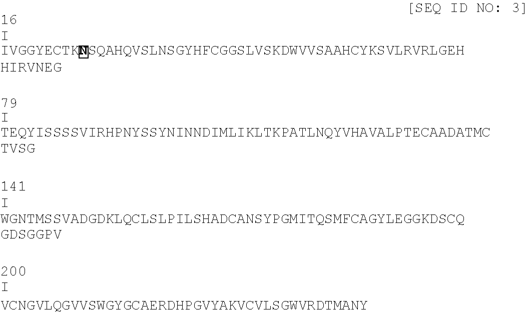
or a fragment thereof which exhibits an antimicrobial activity.

6. A method according to claim 3 wherein the polypeptide having serine protease activity does not comprise lysine at position 160.

7. A method according to claim 6 wherein the polypeptide having serine protease activity comprises or consists of the amino acid sequence of SEQ ID NO:4 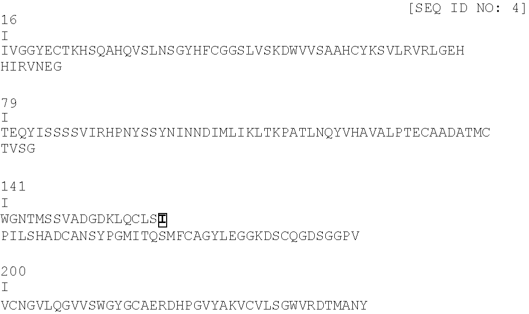
or a fragment thereof which exhibits an antimicrobial activity.

8. A method according to claim 3 wherein the polypeptide having serine protease activity is a variant of SEQ ID NO:1, comprising one or more mutated amino acids selected from the group consisting of amino acid positions:
E21, H25, H29, V47, K49, D50, L63, H71, H72, R74, N76, T79, Y82, S85, S87, N98, I99, V121, M135, V138, M145, V148, D150, K154, L160, M175, 5179, A183, L185, V212, Y217, P225, A229, V233, L234, V238, M242, N244, and/or Y245.
or a fragment thereof which exhibits an antimicrobial activity,
wherein the amino acid numbering is according to Protein Data Bank [PDB] entry ‘2EEK!’.

9. A method according to claim 8 wherein the polypeptide having serine protease activity is a variant of SEQ ID NO:1, comprising one or more mutated amino acids selected from the group consisting of:
E21T, H25Y, H29(Y/N), V47I, K49E, D50Q, L63I, H71D, H72N, R74(K/E), N76(T/L), T79(S/N), Y82F, S85A, S87(K/R), S89R, N98T, I99L, V121I, M135Q, V138I, M145(T/L/V/E/K), V148G, D150S, K154(T/V), L160(I/A), M175(K/Q), S179N, A183V, L185G, V212I, Y217(D/H/S), P225Y, A229V, V233N, L234Y, V238I, M242I, N244S, and/or Y245N.
or a fragment thereof which exhibits an antimicrobial activity,
wherein the amino acid numbering is according to Protein Data Bank [PDB] entry ‘2EEK!’.

10. A method according to claim 3 wherein the polypeptide having serine protease activity is a variant of SEQ ID NO:2, comprising one or more mutated amino acids selected from the group consisting of amino acid positions:
E25, H29, H33, V49, K51, D52, L65, H72, H73, R75, N77, T80, Y83, S86, S88, N99, I100, V122, M134, V137, M144, V147, D149, K152, L158, M173, S177, A181, L184, V208, Y213, P221, A225, V229, L230, V234, M238, N240, and/or Y241.

11. A method according to claim 10 wherein the polypeptide having serine protease activity is a variant of SEQ ID NO:2, comprising one or more mutated amino acids selected from the group consisting of:
E25T, H29Y, H33(Y/N), V49I, K51E, D52Q, L65I, H72D, H73N, R75(K/E), N77(T/L), T80(S/N), Y83F, S86A, S88(K/R), N99T, I100L, V122I, M134Q, V137I, M144(T/L/V/E/K), V147G, D149S, K152(T/V), L158(I/A), M173(K/Q), S177N, A181V, L184G, V208I, Y213(D/H/S), P221Y, A225V, V229N, L230Y, V234I, M238I N240S, and/or Y241N.

12. A method according to claim 1 wherein the polypeptide having serine protease activity comprises or consists of an amino acid sequence as defined in Table 1 or 2.

13. A method according to claim 1 wherein the polypeptide having serine protease activity comprises or consists of the amino acid of SEQ ID NO:1 with one of the following defined mutations:
(a) N244S, Y245N, S87K (“EZA-002”);
(b) K154T (“EZA-003”);
(c) K154L (“EZA-004”);
(d) K154V (“EZA-005”);
(e) K154E (“EZA-006”);
(f) N98T (“EZA-007”);
(g) I99L (“EZA-008”);
(h) L185G, P225Y (“EZA-009”);
(i) V212I (“EZA-0010”);
(j) Y217D, M175K (“EZA-011”);
(k) Y217H (“EZA-012”);
(l) Y217S (“EZA-013”);
(m) A229V (“EZA-014”);
(n) H25Y (“EZA-015”);
(o) H25N (“EZA-016”);
(p) H29Y (“EZA-017”);
(q) H71D (“EZA-018”);
(r) H72N (“EZA-019”);
(s) R74K (“EZA-020”);
(t) R74E (“EZA-021”);
(u) N76T (“EZA-022”);
(v) N76L, Y82F (“EZA-023”);
(w) T79S (“EZA-0024”);
(x) T79N (“EZA-025”);
(y) K49E, D50Q (“EZA-026”);
(z) S87R (“EZA-027”);
(aa) E21T, H71D, D150S, K154V (“EZA-028”);
(bb) S179N, V233N (“EZA-029”);
(cc) M135Q (“EZA-030”);
(dd) M145K, V148G (“EZA-031”);
(ee) M175Q (“EZA-032”);
(ff) L63I, S85A (“EZA-033”);
(gg) L160I (“EZA-034”);
(hh) V138I, L160A, A183V (“EZA-035”);
(ii) V121I (“EZA-036”);
(jj) V47I, V238I, M242I (“EZA-037”);
(kk) V238I (“EZA-038”); and
(11) L234Y (“EZA-039”)
or a fragment thereof which exhibits an antimicrobial activity,
wherein the amino acid numbering is according to Protein Data Bank [PDB] entry ‘2EEK!’.

14. A method according to claim 1 wherein the polypeptide having serine protease activity comprises or consists of the amino acid of SEQ ID NO:1 with one of the following defined mutations:
(a) H25N, N76T
(b) H25N, H29Y
(c) H25N, M135Q
(d) H29Y, T79N, M135Q
(e) I99L, V121I, L160I, Y217H
(f) V121I, L160I
(g) H72N, R74E, S87K
(h) H25N, M135Q, Y217H
(i) T79N, V121I, V212I
(j) H29Y, N76T, I99L, M135Q
(k) K49E, D50Q, N76L, Y82F, S179N, V233N
(l) M145K, V148G, N76L, Y82F, S179N, V233N
(m) H25N, N76T, S87K, K154T
(n) H25Q
(o) H25D
(p) H25S
(q) K24E, H25N
(r) Y97N
(s) N100D
(t) A120S, A122S
(u) M135E
(v) V204Q, A122S
(w) T79D
(x) R74D
(y) K49E
(z) K49S, D50Q
(aa) D50Q
(bb) Q178D
(cc) S87R
or a fragment thereof which exhibits an antimicrobial activity,
wherein the amino acid numbering is according to Protein Data Bank [PDB] entry ‘2EEK!’.

15. A method according to claim 1 wherein the polypeptide having serine protease activity comprises or consists of the amino acid of a naturally-occurring serine protease.

16. A method according to claim 15 wherein the polypeptide having serine protease activity comprises or consists of the amino acid of SEQ ID NO:1.

17. A method according to claim 1 wherein the activation peptide comprises or consist of the amino acid sequence selected from the following group:
[SEQ ID NO: 5]
(a) MEEDK;
[SEQ ID NO: 6]
(b) MTEEDK;
[SEQ ID NO: 7]
(c) MFAEEDK;
[SEQ ID NO: 8]
(d) MVFAEEDK;
[SEQ ID NO: 9]
(e) MAFAEEDK;
and
[SEQ ID NO: 10]
(f) MGAVFAEEDK.

18. A method according to claim 1 wherein in step (a) the nucleic acid molecule encoding a trypsinogen polypeptide is in an expression vector suitable for use in Escherichia coli.

19. A method according to claim 18 wherein the expression vector is E3.

20. A method according to claim 1 wherein the host cell in step (a) is a bacterial host cell (such as Escherichia coli and Pseudoalteromonas haloplanktis).

21. A method according to claim 20 wherein the host cell in step (a) is an Escherichia coli host cell (such as BL21(E3), BL21(DE3), BL21 Star (DE3), ArcticExpress (DE3) and HMS174 cells).

22. A method according to claim 20 wherein the host cell in step (a) is an Escherichia coli host cell of strain ArcticExpress (DE3).

23. A method according to claim 1 wherein the host cell in step (a) is a yeast host cell (such as Pichia pastoris).

24. A method according to claim 1 wherein in step (c) refolding the polypeptide into a bioactive form comprises contacting the polypeptide with a PBS/glycerol buffer.

25. A method according to claim 24 wherein the PBS/glycerol buffer is 1×PBS, 10% glycerol, pH 7.4.

26. A method according to claim 1 wherein in step (c) no inhibitor of autoproteolysis is present (such as benzamidin).

27. A method according to claim 1 wherein in step (d) Atlantic cod trypsin is used to activate the zymogen polypeptide.

28. A method according to claim 1 wherein the specific activity of the activated polypeptide produced in step (d) is at least 20 U/mg, for example at least 30 U/mg, 40 U/mg, 50 U/mg, or at least 60 U/mg.

29. A method according to claim 1 wherein the quantity of the activated polypeptide produced in step (d) is at least 0.1 mg, for example at least 0.5 mg, 1 mg, 2 mg, 3 mg, 5 mg, or 10 mg.

30. An isolated polypeptide having serine protease activity obtainable by a method according to claim 1.

31. An isolated polypeptide according to claim 30 wherein the polypeptide exhibits trypsin activity.

32. An isolated polypeptide according to claim 30 or 31 wherein the polypeptide comprises or consists of an amino acid sequence which shares at least 70% sequence identity with amino acid sequence of SEQ ID NO:1, for example at least 80%, 85%, 90%, 95%, 95%, 97%, 98% or 99% sequence identity, or a fragment thereof which exhibits an antimicrobial activity.

33. An isolated polypeptide according to claim 32 wherein the polypeptide does not comprise histidine at position 25.

34. An isolated polypeptide according to claim 33 comprising or consisting of the amino acids sequence of SEQ ID NO:2, or a fragment thereof which exhibits an antimicrobial activity.

35. An isolated polypeptide according to claim 32 wherein the polypeptide having serine protease activity does not comprise lysine at position 160.

36. An isolated polypeptide according to claim 35 wherein the polypeptide having serine protease activity comprises or consists of the amino acid sequence of SEQ ID NO:3, or a fragment thereof which exhibits an antimicrobial activity.

37. An isolated polypeptide according to claim 30 wherein the polypeptide is a variant of SEQ ID NO:1, comprising one or more mutated amino acids selected from the group consisting of amino acid positions:
E21, H25, H29, V47, K49, D50, L63, H71, H72, R74, N76, T79, Y82, S85, S87, S89, N98, 199, V121, M135, V138, M145, V148, D150, K154, L160, M175, 5179, A183, L185, V212, Y217, P225, A229, V233, L234, V238, M242, N244, and/or Y245,
or a fragment thereof which exhibits an antimicrobial activity,
wherein the amino acid numbering is according to Protein Data Bank [PDB] entry ‘2EEK!’.

38. An isolated polypeptide according to claim 37 wherein the polypeptide having serine protease activity is a variant of SEQ ID NO:1, comprising one or more mutated amino acids selected from the group consisting of:
E21T, H25Y, H29(Y/N), V47I, K49E, D50Q, L63I, H71D, H72N, R74(K/E), N76(T/L), T79(S/N), Y82F, S85A, S87(K/R), S89R, N98T, I99L, V121I, M135Q, V138I, M145(T/L/V/E/K), V148G, D150S, K154(T/V), L160(I/A), M175(K/Q), S179N, A183V, L185G, V212I, Y217(D/H/S), P225Y, A229V, V233N, L234Y, V238I, M242I, N244S, and/or Y245N,
or a fragment thereof which exhibits an antimicrobial activity,
wherein the amino acid numbering is according to Protein Data Bank [PDB] entry ‘2EEK!’.

39. An isolated polypeptide according to claim 30 wherein the polypeptide having serine protease activity is a variant of SEQ ID NO:2, comprising one or more mutated amino acids selected from the group consisting of amino acid positions:
E25, H29, H33, V49, K51, D52, L65, H72, H73, R75, N77, T80, Y83, S86, S88, N99, I100, V122, M134, V137, M144, V147, D149, K152, L158, M173, S177, A181, L184, V208, Y213, P221, A225, V229, L230, V234, M238, N240, and/or Y241.

40. An isolated polypeptide according to claim 39 wherein the polypeptide having serine protease activity is a variant of SEQ ID NO:2, comprising one or more mutated amino acids selected from the group consisting of:
E25T, H29Y, H33(Y/N), V49I, K51E, D52Q, L65I, H72D, H73N, R75(K/E), N77(T/L), T80(S/N), Y83F, S86A, S88(K/R), N99T, I100L, V122I, M134Q, V137I, M144(T/L/V/E/K), V147G, D149S, K152(T/V), L158(I/A), M173(K/Q), S177N, A181V, L184G, V208I, Y213(D/H/S), P221Y, A225V, V229N, L230Y, V234I, M238I, N240S, and/or Y241N.

41. An isolated polypeptide according to claim 30 wherein the polypeptide having serine protease activity comprises or consists of an amino acid sequence as defined in Table 1 or 2.

42. An isolated polypeptide according to claim 30 comprising or consisting of the amino acid of SEQ ID NO:1 with one of the following defined mutations:
(a) N244S, Y245N, S87K (“EZA-002”);
(b) K154T (“EZA-003”);
(c) K154L (“EZA-004”);
(d) K154V (“EZA-005”);
(e) K154E (“EZA-006”);
(f) N98T (“EZA-007”);
(g) I99L (“EZA-008”);
(h) L185G, P225Y (“EZA-009”);
(i) V212I (“EZA-0010”);
(j) Y217D, M175K (“EZA-011”);
(k) Y217H (“EZA-012”);
(l) Y217S (“EZA-013”);
(m) A229V (“EZA-014”);
(n) H25Y (“EZA-015”);
(o) H25N (“EZA-016”);
(p) H29Y (“EZA-017”);
(q) H71D (“EZA-018”);
(r) H72N (“EZA-019”);
(s) R74K (“EZA-020”);
(t) R74E (“EZA-021”);
(u) N76T (“EZA-022”);
(v) N76L, Y82F (“EZA-023”);
(w) T79S (“EZA-0024”);
(x) T79N (“EZA-025”);
(y) K49E, D50Q (“EZA-026”);
(z) S87R (“EZA-027”);
(aa) E21T, H71D, D150S, K154V (“EZA-028”);
(bb) S179N, V233N (“EZA-029”);
(cc) M135Q (“EZA-030”);
(dd) M145K, V148G (“EZA-031”);
(ee) M175Q (“EZA-032”);
(ff) L63I, S85A (“EZA-033”);
(gg) L160I (“EZA-034”);
(hh) V138I, L160A, A183V (“EZA-035”);
(ii) V121I (“EZA-036”);
(jj) V47I, V238I, M242I (“EZA-037”);
(kk) V238I (“EZA-038”); and
(ll) L234Y (“EZA-039”)
or a fragment thereof which exhibits an antimicrobial activity,
wherein the amino acid numbering is according to Protein Data Bank [PDB] entry ‘2EEK!’.

43. An isolated polypeptide according to claim 30 comprising or consisting of the amino acid of SEQ ID NO:1 with one of the following defined mutations:
(a) H25N, N76T
(b) H25N, H29Y
(c) H25N, M135Q
(d) H29Y, T79N, M135Q
(e) I99L, V121I, L160I, Y217H
(f) V121I, L160I
(g) H72N, R74E, S87K
(h) H25N, M135Q, Y217H
(i) T79N, V121I, V212I
(j) H29Y, N76T, I99L, M135Q
(k) K49E, D50Q, N76L, Y82F, S179N, V233N
(l) M145K, V148G, N76L, Y82F, S179N, V233N
(m) H25N, N76T, S87K, K154T
(n) H25Q
(o) H25D
(p) H25S
(q) K24E, H25N
(r) Y97N
(s) N100D
(t) A120S, A122S
(u) M135E
(v) V204Q, A122S
(w) T79D
(x) R74D
(y) K49E
(z) K49S, D50Q
(aa) D50Q
(bb) Q178D
(cc) S87R
or a fragment thereof which exhibits an antimicrobial activity,
wherein the amino acid numbering is according to Protein Data Bank [PDB] entry ‘2EEK!’.

44. An isolated polypeptide according to claim 30 wherein the polypeptide comprises or consists of the amino acid of a naturally-occurring serine protease.

45. An isolated polypeptide according to claim 44 wherein the polypeptide comprises or consists of the amino acid of SEQ ID NO:1.

46. An isolated polypeptide according to claim 30 which exhibits improved stability relative to the trypsin I isolated from Atlantic cod (Gadus morhua).

47. An isolated polypeptide according to claim 46 wherein the trypsin I isolated from Atlantic cod has the amino acid sequence of SEQ ID NO: 1.

48. An isolated polypeptide according to claim 46 which exhibits improved thermal stability relative to the trypsin polypeptide of trypsin I isolated from Atlantic cod.

49. An isolated polypeptide according to claim 48 comprising or consisting of the amino acid of SEQ ID NO:1 with one of the following defined mutations:
(a) K154E (“EZA-006”);
(b) N98T (“EZA-007”);
(c) I99L (“EZA-008”);
(d) V212I (“EZA-0010”);
(e) Y217D, M175K (“EZA-011”);
(f) Y217H (“EZA-012”);
(g) A229V (“EZA-014”);
(h) H25Y (“EZA-015”);
(i) H25N (“EZA-016”);
(j) H72N (“EZA-019”);
(k) R74E (“EZA-021”);
(l) N76L, Y82F (“EZA-023”);
(m) T79N (“EZA-025”);
(n) K49E, D50Q (“EZA-026”);
(o) S87R (“EZA-027”);
(p) E21T, H71D, D150S, K154V (“EZA-028”);
(q) S179N, V233N (“EZA-029”);
(r) M135Q (“EZA-030”);
(s) M145K, V148G (“EZA-031”);
(t) L63I, S85A (“EZA-033”);
(u) L160I (“EZA-034”);
(v) V138I, L160A, A183V (“EZA-035”);
(w) V121I (“EZA-036”);
(x) V47I, V238I, M242I (“EZA-037”); and
(y) L234Y (“EZA-039”)
or a fragment thereof which exhibits an antimicrobial activity,
wherein the amino acid numbering is according to Protein Data Bank [PDB] entry ‘2EEK!’.

50. An isolated polypeptide according to claim 30 which exhibits improved autoproteolytic stability relative to the trypsin I isolated from Atlantic cod.

51. An isolated polypeptide according to claim 50 comprising or consisting of the amino acid of SEQ ID NO:1 with one of the following defined mutations:
(a) I99L (“EZA-008”);
(b) V212I (“EZA-0010”);
(c) Y217D, M175K (“EZA-011”);
(d) Y217H (“EZA-012”);
(e) A229V (“EZA-014”);
(f) H25Y (“EZA-015”);
(g) H25N (“EZA-016”);
(h) H29Y (“EZA-017”);
(i) H72N (“EZA-019”);
(j) R74E (“EZA-021”);
(k) N76T (“EZA-022”);
(l) N76L, Y82F (“EZA-023”);
(m) T79S (“EZA-0024”);
(n) T79N (“EZA-025”);
(o) K49E, D50Q (“EZA-026”);
(p) S87R (“EZA-027”);
(q) E21T, H71D, D150S, K154V (“EZA-028”);
(r) S179N, V233N (“EZA-029”);
(s) M135Q (“EZA-030”);
(t) M145K, V148G (“EZA-031”);
(u) L63I, S85A (“EZA-033”);
(v) L160I (“EZA-034”);
(w) V138I, L160A, A183V (“EZA-035”);
(x) V121I (“EZA-036”); and
(y) V47I, V238I, M242I (“EZA-037”).
or a fragment thereof which exhibits an antimicrobial activity,
wherein the amino acid numbering is according to Protein Data Bank [PDB] entry ‘2EEK!’.

52. An isolated polypeptide according to claim 30 which exhibits an improved Kcat relative to trypsin I isolated from Atlantic cod.

53. An isolated polypeptide according to claim 52 comprising or consisting of the amino acid of SEQ ID NO:1 with one of the following defined mutations:
(a) H25N (“EZA-016”);
(b) H29Y (“EZA-017”);
(c) H71D (“EZA-018”);
(d) H72N (“EZA-019”);
(e) N76T (“EZA-022”);
(f) N76L, Y82F (“EZA-023”);
(g) M145K, V148G (“EZA-031”);
(h) M175Q (“EZA-032”);
(i) L63I, S85A (“EZA-033”);
(j) L160I (“EZA-034”);
(k) V138I, L160A, A183V (“EZA-035”);
(l) V47I, V238I, M242I (“EZA-037”); and
(m) V238I (“EZA-038”),
or a fragment thereof which exhibits an antimicrobial activity,
wherein the amino acid numbering is according to Protein Data Bank [PDB] entry ‘2EEK!’.

54. An isolated polypeptide according to claim 30 which exhibits an improved Km relative to trypsin I isolated from Atlantic cod.

55. An isolated polypeptide according to claim 54 comprising or consisting of the amino acid of SEQ ID NO:1 with one of the following defined mutations:
(a) K154T (“EZA-003”);
(b) I99L (“EZA-008”);
(c) V212I (“EZA-0010”);
(d) Y217D, M175K (“EZA-011”);
(e) Y217S (“EZA-013”);
(f) A229V (“EZA-014”);
(g) H25Y (“EZA-015”);
(h) K49E, D50Q (“EZA-026”);
(i) E21T, H71D, D150S, K154V (“EZA-028”); and
(j) M135Q (“EZA-030”),
or a fragment thereof which exhibits an antimicrobial activity,
wherein the amino acid numbering is according to Protein Data Bank [PDB] entry ‘2EEK!’.

56. An isolated polypeptide according to claim 30 which exhibits an improved specificity constant (Kcat/Km) relative to trypsin I isolated from Atlantic cod.

57. An isolated polypeptide according to claim 56 comprising or consisting of the amino acid of SEQ ID NO:1 with one of the following defined mutations:
(a) K154T (“EZA-003”);
(b) Y217D, M175K (“EZA-011”);
(c) Y217S (“EZA-013”);
(d) A229V (“EZA-014”); and
(e) M135Q (“EZA-030”);
or a fragment thereof which exhibits an antimicrobial activity,
wherein the amino acid numbering is according to Protein Data Bank [PDB] entry ‘2EEK!’.

58. An isolated polypeptide according to claim 30 which is non-glycosylated.

59. An isolated polypeptide according to claim 30 comprising or consisting of L-amino acids.

60. An isolated polypeptide according to claim 30 comprising one or more amino acids that are modified or derivatised.

61. An isolated polypeptide according to claim 60 wherein the one or more amino acids are modified or derivatised by PEGylation, amidation, esterification, acylation, acetylation and/or alkylation.

62. An isolated nucleic acid molecule which encodes a polypeptide according to claim 30.

63. An expression vector comprising a nucleic acid molecule according to claim 62.

64. An expression vector according to claim 63 suitable for use in Escherichia coli.

65. A microbial host cell comprising a nucleic acid molecule according to claim 62.

66. A host cell according to claim 65 wherein the host cell is a bacterial host cell (such as Escherichia coli and Pseudoalteromonas haloplanktis).

67. A host cell according to claim 59 wherein the host cell is an Escherichia coli host cell (such as BL21(E3), BL21(DE3), BL21 Star (DE3), ArcticExpress (DE3) and HMS174 cells).

68. A host cell according to claim 67 wherein the host cell in step (a) is an Escherichia coli host cell of strain ArcticExpress (DE3).

69. A host cell according to claim 65 wherein the host cell is a yeast host cell (such as Pichia pastoris).

70. A therapeutic composition comprising a polypeptide according to claim 30 together with a pharmaceutically acceptable excipient, diluent, carrier, buffer or adjuvant.

71. A therapeutic composition according to claim 70 suitable for administration via a route selected from the group consisting of oral, nasal, pulmonar, buccal, topical, ocular, parenteral (intravenous, subcutaneous, intratechal and intramuscular), vaginal and rectal.

72. A therapeutic composition according to claim 70 wherein the polypeptide is provided in a form suitable for delivery to the mucosa of the mouth and/or oropharynx.

73. A therapeutic composition according to claim 72 wherein the polypeptide is provided in a mouth spray, lozenge, pastille, chewing gum or liquid.

74. A therapeutic composition according to claim 73 wherein the polypeptide is provided in a mouth spray.

75. A polypeptide according to claim 30 for use in medicine.

76. A polypeptide according to claim 30 for use in the treatment or prevention in a subject of a disorder or condition selected from the groups consisting of microbial infections, inflammation and wounds.

77. A polypeptide for use according to claim 76 wherein the disorder or condition is a microbial infection.

78. A polypeptide for use according to claim 77 wherein the microbial infection is selected from the group consisting of bacterial infections, viral infections, fungal infections, parasitic infections and yeast infections.

79. A polypeptide for use according to claim 78 wherein the microbial infection is a bacterial infection.

80. A polypeptide for use according to claim 79 wherein the microbial infection comprises formation of a biofilm.

81. A polypeptide for use according to claim 79 wherein the microbial infection is periodontal disease.

82. A polypeptide for use according to claim 77 wherein the microbial infection is a viral infection.

83. A polypeptide for use according to claim 82 wherein the viral infection is selected from the group consisting of the common cold and influenza.

84. A polypeptide for use according to claim 82 wherein the viral infection is caused by an enterovirus (such as a human rhinovirus or Coxsackie A virus).

85. A polypeptide for use according to claim 82 wherein the viral infection is caused by a herpes simplex virus.

86. A polypeptide for use to claim 77 wherein the microbial infection is a fungal infection.

87. A polypeptide for use according to claim 86 wherein the fungal infection is selected from the group consisting of tinea pedis (athlete's foot) and candidiasis (thrush).

88. A polypeptide for use according to claim 76 wherein the subject has or is susceptible to an immunodeficiency.

89. A polypeptide for use according to claim 88 wherein the immunodeficiency is a secondary or acquired immunodeficiency, for example the subject is receiving treatment with an immunosuppressant therapy.

90. A polypeptide for use according to claim 89 wherein the immunodeficiency is naturally-occurring, for example the immunodeficiency is due to a primary immunodeficiency, a cancer (such as leukemia, lymphoma, multiple myeloma), chronic infection (such as acquired immunodeficiency syndrome or AIDS), malnutrition and/or aging.

91. A polypeptide for use according to claim 76 wherein the microbial infection is of the mouth and/or oropharynx.

92. A polypeptide for use according to claim 76 wherein the subject is an athlete (for example, a marathon runner).

93. A polypeptide for use according to claim 76 wherein the disorder or condition is an inflammatory disorder or condition.

94. A polypeptide for use according to claim 93 wherein the inflammatory disorder or condition is selected from the group consisting of pain, acute inflammation, chronic inflammation, arthritis, inflamed joints, bursitis, osteoarthritis, rheumatoid arthritis, juvenile rheumatoid arthritis, septic arthritis, fibromyalgia, systemic lupus erythematosus, phlebitis, tendinitis, rash, psoriasis, acne, eczema, facial seborrheic eczema, and eczema of the hands, face or neck.

95. A polypeptide for use according to claim 76 wherein the disorder or condition is a wound.

96. A polypeptide for use according to claim 95 wherein the wound is selected from acute traumas (including burns), topical ulcers, scars, keloids, boils and warts.

97. A polypeptide for use according to claim 95 wherein the polypeptide is for debridement (i.e. removing infected, dead or peeling skin from otherwise healthy skin) and/or removal of fibrin clots.

98. A polypeptide for use according to claim 76 wherein the polypeptide is for use in combination with one or more additional active agents.

99. A polypeptide for use according to claim 98 wherein the additional active agents are selected from the group consisting of antimicrobial agents (including antibiotics, antiviral agents and anti-fungal agents), anti-inflammatory agents (including steroids and non-steroidal anti-inflammatory agents) and antiseptic agents.

100. Use of a polypeptide according to claim 30 in the preparation of a medicament for the treatment or prevention in a subject of a disorder or condition selected from the groups consisting of microbial infections, inflammation and wounds.

101. A method for the treatment or prevention in a subject of a disorder or condition selected from the groups consisting of microbial infections, inflammation and wounds, the method comprising administering an effective amount of a polypeptide according to claim 30 to a subject in need thereof.

102. Use of a polypeptide according to claim 30 as a cosmetic therapy in a subject.

103. The use according to claim 102 wherein the cosmetic therapy provides one or more of the following effects to the subject:
(a) exfoliating of skin (removal of dead and/or peeling skin cells);
(b) protecting against the breakdown of collagen and elastin in skin;
(c) a comedolytic effect;
(d) reducing or preventing glabellar (frown) lines; and/or
(e) promoting hair growth.

104. A method of cosmetic therapy in a subject comprising administering an effective amount of a polypeptide according to claim 30 to a subject.

105. A method according to claim 104 wherein the cosmetic therapy provides one or more of the following effects to the subject:
(a) exfoliating of skin (removal of dead and/or peeling skin cells);
(b) protecting against the breakdown of collagen and elastin in skin;
(c) a comedolytic effect;
(d) reducing or preventing glabellar (frown) lines; and/or
(e) promoting hair growth.

106. Use of a polypeptide according to claim 30 as an industrial agent.

107. The use according to claim 106 wherein the industrial agent is:
(a) a textile treatment agent;
(b) a biocatalyst (e.g. in the organic synthesis of pharmaceuticals)
(c) a cleaning/hygiene agent (e.g. a detergent);
(d) an environmental bioremediation agent (e.g. to reduce contamination);
(e) a molecular biology agent; and
(f) a food product treatment agent (e.g. in dairy manufacturing).

108. A method for the production of a recombinant polypeptide having serine protease activity substantially as herein defined with reference to the description.

109. An isolated polypeptide substantially as herein defined with reference to the description.

110. A polypeptide for use in medicine substantially as herein defined with reference to the description.

111. Use of a polypeptide substantially as herein defined with reference to the description.


SEQUENCE LISTING (18 sequences)

SEQ ID NO: 1 - protein, 222 aa, Gadus morhua
IVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 2 - protein, 241 aa, Gadus morhua
MKSLIFVLLLGAVFAEEDKIVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 3 - protein, 222 aa, Artificial Sequence
  Synthetic peptide
IVGGYECTKNSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 4 - protein, 222 aa, Artificial Sequence
  Synthetic peptide
IVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSIPILSHADCANSYPGMITQSMFCAGYLEGGKDSCQGDSGGPVVCNGVLQGVVSWGYGCAERDHPGVYAKVCVLSGWVRDTMANY

SEQ ID NO: 5 - protein, 5 aa, Artificial Sequence
  Synthetic peptide
MEEDK

SEQ ID NO: 6 - protein, 6 aa, Artificial Sequence
  Synthetic peptide
MTEEDK

SEQ ID NO: 7 - protein, 7 aa, Artificial Sequence
  Synthetic peptide
MFAEEDK

SEQ ID NO: 8 - protein, 8 aa, Artificial Sequence
  Synthetic peptide
MVFAEEDK

SEQ ID NO: 9 - protein, 8 aa, Artificial Sequence
  Synthetic peptide
MAFAEEDK

SEQ ID NO: 10 - protein, 10 aa, Artificial Sequence
  Synthetic peptide
MGAVFAEEDK

SEQ ID NO: 11 - DNA, 678 bp, Gadus morhua
gaagaagataaaatcgttggcggctatgaatgcacgaaacactcgcaggcacaccaggtctcactgaacagcggttaccacttttgcggcggtagtctggttagcaaagattgggttgttagtgcggcccattgctataaaagcgtgctgcgtgttcgcctgggcgaacatcacattcgtgtgaatgaaggcaccgaacagtacattagctctagtagcgttatccgccatccgaactactctagttacaacatcaacaacgatatcatgctgatcaaactgaccaaaccggcgacgctgaaccagtatgtgcacgccgttgcactgccgaccgaatgcgcagcggatgcaaccatgtgtaccgtgagcggctggggtaatacgatgagctctgttgcggatggcgataaactgcagtgcctgtctctgccgattctgagtcatgcggattgtgccaactcttatccgggcatgatcacgcagagcatgttttgcgccggttacctggaaggcggtaaagatagctgccagggtgattctggcggtccggtggtttgtaacggcgttctgcagggtgtggttagctggggctacggttgtgcagaacgtgatcacccgggtgtctatgctaaagtctgtgtgctgtcgggctgggtccgtgatacgatggcgaactat

SEQ ID NO: 12 - DNA, 684 bp, Artificial Sequence
  Synthetic oligonucleotide
atggaagaagataaaatcgttggcggctatgaatgcacgaaacactcgcaggcacaccaggtctcactgaacagcggttaccacttttgcggcggtagtctggttagcaaagattgggttgttagtgcggcccattgctataaaagcgtgctgcgtgttcgcctgggcgaacatcacattcgtgtgaatgaaggcaccgaacagtacattagctctagtagcgttatccgccatccgaactactctagttacaacatcaacaacgatatcatgctgatcaaactgaccaaaccggcgacgctgaaccagtatgtgcacgccgttgcactgccgaccgaatgcgcagcggatgcaaccatgtgtaccgtgagcggctggggtaatacgatgagctctgttgcggatggcgataaactgcagtgcctgtctattccgattctgagtcatgcggattgtgccaactcttatccgggcatgatcacgcagagcatgttttgcgccggttacctggaaggcggtaaagatagctgccagggtgattctggcggtccggtggtttgtaacggcgttctgcagggtgtggttagctggggctacggttgtgcagaacgtgatcacccgggtgtctatgctaaagtctgtgtgctgtcgggctgggtccgtgatacgatggcgaactattaa

SEQ ID NO: 13 - protein, 229 aa, Artificial Sequence
  Synthetic peptide
MFAEEDKIVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 14 - protein, 230 aa, Artificial Sequence
  Synthetic peptide
MVFAEEDKIVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGNNDIMLIKLTKPATLNQYVHAVALPTECAADATMCTVSGWGNTMSSVADGCNGVLQGVVSWGYGCAERDHPGVYAKVCVLSGWVRDTMANY

SEQ ID NO: 15 - protein, 219 aa, Artificial Sequence
  Synthetic peptide
MAFAEEDKIVGGYECTKHSQAHQVSLNSGYHFCGGSLVSMAFAEEDKIVGGYECTKHSQAHQVSLNSGYHFCGGSLVSNNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 16 - protein, 232 aa, Artificial Sequence
  Synthetic peptide
MGAVFAEEDKIVGGYECTKHSQAHQVSLNSGYHFCGGSLVSKDWVVSAAHCYKSVLRVRLGEHHIRVNEGTEQYISSSSVIRHPNYSSYNINNDIMLIKLTKPATLNQYVHAVALPTECAADATMCTVSGWGNTMSSVADGDKLQCLSLPILSHADCANSYPGMITQSMFCAGYLEGGKDSCQGDSGGPVVCNGVLQGVVSWGYGCAERDHPGVYAKVCVLSGWVRDTMANY

SEQ ID NO: 17 - protein, 6 aa, Artificial Sequence
  Synthetic peptide
FAEDDK

SEQ ID NO: 18 - protein, 6 aa, Artificial Sequence
  Synthetic peptide
FAEDDK